(19) United States
(12) Patent Application Publication
(10) Pub. No.: US 2022/0168230 A1
(43) Pub. Date: Jun. 2, 2022

(54) NANOPARTICLE MEDIATED THERAPY

(71) Applicant: Yale University, New Haven, CT (US)

(72) Inventors: Jiangbing Zhou, Cheshire, CT (US); Kevin Sheth, Madison, CT (US); Gang Deng, Hubei (CN); Shenqi Zhang, New Haven, CT (US); Zeming Chen, New Haven, CT (US); Chao Ma, Beijing (CN)

(21) Appl. No.: 17/434,297

(22) PCT Filed: Feb. 26, 2020
(86) PCT No.: PCT/US2020/019925

§ 371 Date: Aug. 26, 2021

Related U.S. Application Data
(60) Provisional application No. 62/810,605, filed on Feb. 26, 2019.

(51) Int. Cl. A61K 9/51; A61K 31/64; A61K 31/137; A61K 31/519; A61K 31/343; A61K 47/26

(52) U.S. Cl. CPC A61K 9/5123; A61K 31/64; A61K 47/26; A61K 31/519; A61K 31/343; A61K 31/137

(57) ABSTRACT

At least five classes of MNP-based compounds have been demonstrated to form supramolecular particles for effective delivery by injection or topically of different types of therapeutic, prophylactic, or diagnostic agents. These compounds are isolated from natural sources such as plants. Exemplary MNP-based compounds, from which synthetic analogs or derivatives are made and appreciated to function similarly, e.g., capable of forming supramolecular particles include diterpene resin acids (e.g., abietic acid and pimaric acid), phytosterols (e.g., stigmasterol and β-sitosterol), lupane-type pentacyclic triterpenes (e.g., lupeol and betulinic acid), oleanane-type pentacyclic tritepenes (e.g., glycyrrhetic acid and sumaresinolic acid), and lanostane-type triterpenes and derivatives (e.g., dehydrotrametenolic acid and poricoic acid A). In some cases the MNP-based compounds are therapeutically effective in the absence of added therapeutic, prophylactic or diagnostic agent. Betulinic acid (BA) NPs were capable of efficiently penetrating ischemic brains and effectively promoting functional recovery as antioxidant agents.

### CROSS-REFERENCE TO RELATED APPLICATIONS

This application claims the benefit of and priority to U.S. Provisional Application No. 62/810,605 filed Feb. 26, 2019, which is hereby incorporated by reference in its entirety.

### STATEMENT REGARDING FEDERALLY SPONSORED RESEARCH

This invention was made with Government support under NIH Grant No. NS095817. The Government has certain rights in the invention.

### FIELD OF THE INVENTION

The present application is generally in the field of nanoparticle mediated therapy, for example, for drug delivery into the brain to treat edema and oxidative damage in conditions such as stroke.

### BACKGROUND OF THE INVENTION

Carriers are frequently used to facilitate delivery of drugs to a specific location or to increase half-life of the drug, penetration into a particular tissue, or release over time or at specific times. Synthetic carriers such as polylactide-co-glycolide (“PLGA”) are well known for their controlled drug delivery properties.

However, even particles such as PLGA can cause localized irritation or inflammation, and must be manufactured using organic solvents, which can lead to loss of activity of the encapsulated drug and regulatory compliance issues. Therefore alternatives which are biocompatible and yet form particles for drug delivery by nanoprecipitation rather than through solvent techniques are highly desirable.

It is also a goal to provide materials to form nanoparticles which have advantageous properties in penetration into tissues such as the brain where significant barriers preclude systemic delivery. The brain has two barriers, the blood brain barrier and the barriers at the surface of the brain cells and endothelial cells, lining the interstitial spaces.

It is therefore an object of the present invention to provide nanoparticulate materials which can be used to form drug delivery particles by nanoprecipitation.

It is a further object of the present invention to provide nanoparticulate materials with improved penetration of the brain, which can provide improved treatments for ischemia, especially those resulting from stoke.

It is another object of the present invention to provide combination therapies that contain therapeutic and/or prophylactic synthetic compounds and medicinal herbal extract materials that can encapsulate and enhance delivery of the agents to the brain.

### SUMMARY OF THE INVENTION

At least five classes of MNP-based compounds have been demonstrated to form supramolecular particles for effective delivery by injection or topically of different types of therapeutic, prophylactic, or diagnostic agents. These compounds are isolated from natural sources such as plants. Exemplary MNP-based compounds, from which synthetic analogs or derivatives are made and appreciated to function similarly, e.g., capable of forming supramolecular particles include diterpene resin acids (e.g., abietic acid and pimaric acid), phytosterols (e.g., stigmasterol and β-sitosterol), lupane-type pentacyclic triterpenes (e.g., lupeol and betulinic acid), oleanane-type pentacyclic tritepenes (e.g., glycyrrhetic acid and sumaresinolic acid), and lanostane-type triterpenes and derivatives (e.g., dehydrotrametenolic acid and poricoic acid A). In some cases the MNP-based compounds are therapeutically effective in the absence of added therapeutic, prophylactic or diagnostic agent.

Betulinic acid (BA) NPs were capable of efficiently penetrating ischemic brains and effectively promoting functional recovery as antioxidant agents in animal models where stroke was induced by middle cerebral artery occlusion (MCAO). BA NPs significantly enhances the delivery of a therapeutic agent such as glyburide, which has an anti-edema effect but a limited ability to penetrate the ischemic brain as determined by positron emission tomography-computed tomography (PET/CT), resulting in therapeutic benefits greater than those achieved by either glyburide or BA NPs alone.

Additional materials identified using the same approach which also formed nanoparticles, include ursolic acid (UA), stigmasterol (ST), sumaresinolic acid (SA), glycyrrhetic acid (GA), dehydrotrametenolic acid (DTA), poricoic acid A (PAA), lupeol (LP), β-sitosterol (BT), and oleanolic acid (OA). NPs containing UA, ST, SA, GA, DTA, PAA, LP, BT, or OA effectively promoted stroke recovery after intravenous administration.

Other neuroprotective agents, such as Tat-NR2B9c, can be used as a payload in the nanoparticles described herein, for treating strokes.

In some forms, the nanoparticles without payload (also referred to as “empty nanoparticles”) exhibit therapeutic effect and can also be used to treat stoke.

In some embodiments, the NPs are in the form of nanospheres, optionally having an average diameter of between about 10 and about 500 nm, preferably between about 20 and about 100 nm. In some embodiments, the NPs are in the form of nanorods, preferably having an average length of between about 100 and about 600 nm, preferably between about 200 and about 400 nm.

These results show that the method is generally useful to identify functional nanomaterials as well as a promising approach to achieving anti-edema and antioxidant combination therapy for ischemic stroke via a simple formulation.

### BRIEF DESCRIPTION OF THE DRAWINGS

FIGS. 1A and 1B. (A) Preparation of ¹¹C-labeled glyburide. (B) Standardized uptake value (SUV) with time for left (normal) and right (ischemia) hemispheres.

FIGS. 2A and 2B. (A) Procedures for nanomaterial isolation from E. ulmoides. (B) Molecular structure of BA.

FIGS. 3A-3E. BA NPs for delivery to a tissue subject to stroke injury. (A) Semi-quantification of BA NPs in the brains isolated from MCAO mice received the indicated treatment. The quantification was performed based on fluorescent imaging. (B) Flow cytometry analysis of the uptake of BA NPs in cells that were engineered to overexpress the indicated surface molecules. (C) Schematic diagram of in vitro BBB transcytosis assay. (D) In vitro analysis of the inhibitory effect of SR141716A on NP transcytosis. (E) Semi-quantification of IR780-loaded BA NPs in the brains isolated from MCAO mice with and without pre-treatment of SR141716A. The quantification was performed based on fluorescent imaging. Intensities of IR780 fluorescence were quantified using Living Image 3.0.

FIG. 4. Quantification of IR780-loaded BA NPs in major organs after intravenous administration to MCAO mice. The quantification was performed based on fluorescent imaging. Mice were euthanized 24 hours after treatment. Images were captured by an IVIS system. Intensities of IR780 fluorescence were quantified using Living Image 3.0.

FIGS. 5A and 5B. Characterization of BA NPs for stroke treatment. (A) Quantification of brain infarction in MCAO mice received treatment of BA NPs at the indicated dose. The quantification was performed using TTC staining. (B) The impact of BA NPs treatment on the Nrf2 pathway.

FIGS. 6A-6D. Characterization of the pharmacological activities of Gly-NPs for stroke treatment. (A) Release of glyburide from Gly-NPs in PBS at 37° C. (B-D) Kaplan-Meier survival analysis (B), infarct volume (C, day 3 after surgery), and neurological scores (D, day 3 after surgery) of MCAO mice receiving the indicated treatments (n=5).

FIGS. 7A and 7B. Characterization of the pharmacological activities of Gly-NPs on stroke and TBI. (A) Treatment with Gly-NPs effectively reduced brain edema. MCAO mice were prepared and received a single injection of PBS, or Gly-NPs at a dose equivalent to 5 μg/kg of glyburide immediately after surgery (n=5). After 24 hours, the mice were sacrificed and the brains were excised, and weighted to obtain the wet weight. Then, the brains were lyophilized for 24 h and weighted to obtain the dry weight. Tissue water content was calculated as: Tissue water (%)=(wet weight-dry weight)/wet weight×100. Glyburide-loaded BA NPs significantly reduced injured volumes in TBI mouse model. (B) Plot of brain volume (percent) for control PBS, free glyburide, BA NPs, and glyburide-loaded BA NPs.

FIGS. 8A-8C. Characterization of the additional nanomaterials. (A) Molecular structures of UA, ST, and OA. (B) UA-, ST-, and OA-NPs enhanced delivery to the ischemic brain. (C) Quantification of infarct volumes in the brains isolated from MCAO mice received treatment of the indicated NPs (n=3). The quantification was performed based on TC imaging.

FIGS. 9A and 9B. Synthesis of BAM for acidity-triggered drug release. (A) Scheme of BAM synthesis. (B) Release of glyburide from BAM-NPs in buffers with pH 7.4 or 6.8.

FIGS. 10A and 10B. AMD3100-conjugated BAM-NPs improved the delivery and efficacy of peptide therapeutic Tat-NR2B9c for stroke treatment. (A) Semi-quantification of BA NPs and BAM NPs in the brains isolated from MCAO mice received the indicated treatment. “BA”: BA NPs; “PBA”:BAM NPs; “PBA-PEG”: (B) Quantification of infarct volume (percent) in the brains isolated from MCAO mice received treatment of the indicated treatments (n=3).

FIGS. 11A and 11B. Characterization of the additional nanomaterials. (A) Molecular structures of SA, GA, OA, UA, DTA, PAA, ST, and LP. (B) Quantification of infarct volumes in the brains isolated from MCAO mice received treatment of the indicated NPs. The quantification was performed based on TTC imaging.

### DETAILED DESCRIPTION OF THE INVENTION

### I. Definitions

The term “medicinal natural product” refers to various classes of natural products from plant, microbial, and animal natural products, usually produced from sequences of metabolic activity, which have traditional or modern medicine values alone or in combination with other agents. Biosynthetic, semi-synthetic, or synthetic analogues or derivatives of medicinal natural product may share similar modes of action to medicinal natural product, which is intended to be encompassed by the present disclosure.

The term “nanoparticle” or “nanoparticulate” refers to a particle of any shape having a diameter from about 1 nm up to, but not including, about 1 micron. Nanoparticles having a spherical shape are generally referred to as “nanospheres”. Nanoparticle or nanoparticulate compositions may have a spherical, hollow, and/or rod shape.

Microparticles may also be formed based on the identified compounds via common techniques to form microparticles. Microparticles generally refer to particles of any shape having a diameter from 1 μm up to a few millimeters. For penetration across GI track, nanoparticles formed from these identified compounds from medicinal natural products are preferred in some embodiment.

The term “supramolecular particle” refers to micro- or nano-particles formed from many molecules of one or more isolated compounds by noncovalent associations.

The term “bioavailability” refers to the proportion of a therapeutic or prophylactic agent that enters the circulation when introduced into the body. It may be measured as a concentration of the delivered agent or substance in the plasma, or indirectly as the level of signal of the substrate that the delivered agent or substance acts on.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, arylalkyl, substituted arylalkyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups. Such alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, arylalkyl, substituted arylalkyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

Except where specifically provided to the contrary, the term “substituted” refers to a structure, e.g., a chemical compound or a moiety on a larger chemical compound, regardless of how the structure was formed. The structure is not limited to a structure made by any specific method.

“Aryl,” as used herein, refers to C₅-C₂₆-membered aromatic, fused aromatic, fused heterocyclic, or biaromatic ring systems. Broadly defined, “aryl,” as used herein, includes 5-, 6-, 7-, 8-, 9-, 10-, 14-, 18-, and 24-membered single-ring aromatic groups, for example, benzene, naphthalene, anthracene, phenanthrene, chrysene, pyrene, corannulene, coronene, etc.

“Aryl” further encompasses polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle,” “heterocyclic” and “heterocyclyl” are used interchangeably, and refer to a cyclic radical attached via a ring carbon or nitrogen atom of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Examples of heterocycles include, but are not limited to piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₂₆-membered aromatic, fused aromatic, biaromatic ring systems, or combinations thereof, in which one or more carbon atoms on one or more aromatic ring structures have been substituted with an heteroatom. Suitable heteroatoms include, but are not limited to, oxygen, sulfur, and nitrogen. Broadly defined, “heteroaryl,” as used herein, includes 5-, 6-, 7-, 8-, 9-, 10-, 14-, 18-, and 24-membered single-ring aromatic groups that may include from one to four heteroatoms, for example, pyrrole, furan, thiophene, imidazole, oxazole, thiazole, triazole, tetrazole, pyrazole, pyridine, pyrazine, pyridazine and pyrimidine, and the like. The heteroaryl group may also be referred to as “aryl heterocycles” or “heteroaromatics”. “Heteroaryl” further encompasses polycyclic ring systems having two or more rings in which two or more carbons are common to two adjoining rings (i.e., “fused rings”) wherein at least one of the rings is heteroaromatic, e.g., the other cyclic ring or rings can be cycloalkyls, cycloalkenyls, cycloalkynyls, aryls, heterocycles, or combinations thereof. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl”.

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Alkyl,” as used herein, refers to the radical of saturated aliphatic groups, including straight-chain alkyl, alkenyl, or alkynyl groups, branched-chain alkyl, cycloalkyl (alicyclic), alkyl substituted cycloalkyl groups, and cycloalkyl substituted alkyl. In preferred embodiments, a straight chain or branched chain alkyl has 30 or fewer carbon atoms in its backbone (e.g., C₁-C₃₀ for straight chains, C₃-C₃₀ for branched chains), preferably 20 or fewer, more preferably 15 or fewer, most preferably 10 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfoxide, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

“Alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NRR′, wherein R and R′ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CON(R)₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, haloalkyl (such as —CF₃, —CH₂—CF₃, —CCl₃); —CN; —NCOCOCH₂CH₂; —NCOCOCHCH; —NCS; and combinations thereof.

The term “sulfonyl” is represented by the formula

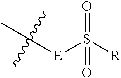

wherein E is absent, or E is alkyl, alkenyl, alkynyl, aralkyl, alkylaryl, cycloalkyl, aryl, heteroaryl, heterocyclyl, wherein independently of E, R represents a hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or E and R taken together with the S atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of E and R can be substituted or unsubstituted amine, to form a “sulfonamide” or “sulfonamido.” The substituted or unsubstituted amine is as defined above.

The term “derivatives” in one or more relevant contexts include replacement of one or more hydrogen, methyl, carboxyl, hydroxyl, or C₂-C₄ alkyl or alkene with one or more of amine, carboxyl, amide, carbonyl, (straight or branched) C₁-C₂₀ alkyl, polyethylene glycol, aryl (including phenyl, indole), C(═O)NR₁R₂ (where R₁ denotes hydrogen, alkyl or aryl; and R₂ denotes heterocyclic unsaturated or saturated radical having 1 to 4 heteroatoms of elements nitrogen, oxygen, and/or sulfur from the group including furanyl, oxazolyl, isooxazolyl, thiophenyl, thiazolyl, isothiazolyl, pyrrolyl, imidazolyl, pyrazolyl, oxadiazolyl, thiadiazoyl, pyridyl, pyrimidinyl, pyrazinyl, pyridazinyl, triazinyl, triazolyl, tetrazolyl, it being possible for the heterocyclic radical to be substituted once or twice, identically or differently, by halogen, C₁˜C₂-alkyl, C₁˜C₄-alkoxy, C₁˜C₄-alkylthio, hydroxy, mercapto, trifluoromethyl, nitro, phenyl, nitrile, carboxy or C₁˜C₄-alkoxycarbonyl). One or more carbons referred to herein may be substituted or unsubstituted.

The term “treating” preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine.

The phrase “therapeutically effective amount” refers to an amount of the therapeutic agent that, when incorporated into and/or onto particles, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation.

The terms “incorporated” and “encapsulated” refers to incorporating, formulating, or otherwise including an active agent into and/or onto a composition that allows for release of such agent in the desired application. The terms contemplate any manner by which a therapeutic agent or other material is incorporated into a polymer matrix, including, for example, attached to a monomer of such polymer (by covalent, ionic, or other binding interaction), physical admixture, enveloping the agent in a coating layer of polymer, and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to—the incorporation of a therapeutic agent or other material and at least one other therapeutic agent or other material in a subject composition. More specifically, the physical form in which any therapeutic agent or other material is encapsulated in polymers may vary with the particular embodiment. For example, a therapeutic agent or other material may be first encapsulated in a microsphere and then combined with the polymer in such a way that at least a portion of the microsphere structure is maintained. Alternatively, a therapeutic agent or other material may be sufficiently immiscible in the polymer that it is dispersed as small droplets, rather than being dissolved, in the polymer.

The term “biocompatible”, as used herein, refers to a material that along with any metabolites or degradation products thereof that are generally non-toxic to the recipient and do not cause any significant adverse effects to the recipient. Generally speaking, biocompatible materials are materials which do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

The term “small molecule”, as used herein, generally refers to an organic molecule that is less than about 1500 g/mol, less than about 1000 g/mol, less than about 800 g/mol, or less than about 500 g/mol. Small molecules are non-polymeric and/or non-oligomeric.

The term “hydrophilic”, as used herein, refers to substances that have strongly polar groups that readily interact with water. The term “hydrophobic”, as used herein, refers to substances that lack an affinity for water, tending to repel and not absorb water as well as not dissolve in or mix with water. The term “lipophilic”, as used herein, refers to compounds having an affinity for lipids. The term “amphiphilic”, as used herein, refers to a molecule combining hydrophilic and lipophilic (hydrophobic) properties.

### II. Compositions

A. MNP-Based Compounds Forming Supramolecular Particles

1. Compounds

At least five classes of MNP-based compounds have been demonstrated to form supramolecular particles for effective delivery of different types of therapeutic, prophylactic, or diagnostic agents. These compounds are isolated from natural sources such as plants. Exemplary MNP-based compounds, from which synthetic analogs or derivatives are made and appreciated to function similarly, e.g., capable of forming supramolecular particles include diterpene resin acids (e.g., abietic acid and pimaric acid), phytosterols (e.g., stigmasterol and β-sitosterol), lupane-type pentacyclic triterpenes (e.g., lupeol and betulinic acid), oleanane-type pentacyclic tritepenes (e.g., glycyrrhetic acid and sumaresinolic acid), and lanostane-type triterpenes and derivatives (e.g., dehydrotrametenolic acid and poricoic acid A).

These compounds are isolated and extracted from natural plant, microbial, or animal products in one or more ways. For example, a crude natural product is heated or boiled in water or an aqueous medium in the presence of one or more superparamagnetic metal oxide nanodots (e.g., superparamagnetic iron oxide (SPIO) nanodots), such that compounds capable of forming supramolecular nanoparticles are associated with the superparamagnetic metal nanodots, the complex of which is further isolated using a magnet. In a second example, a plant, microbial, or animal product is immersed in an appropriate organic solvent such as dichloromethane, chloroform, and ethyl acetate, where the dissolved filtrate is collected to remove undissolvable impurity and to enrich the compounds for forming supramolecular particles. The organic phase filtrate is emulsified in the presence of one or more superparamagnetic metal nanodots (e.g., SPIO nanodots), such that compounds to form supramolecular particles are associated with the superparamagnetic metal nanodots, forming a “complex” that is further isolated using a magnet.

Using either approach, further purification of isolated compounds to separate from the SPIO nanodots usually involves immersing the compound-SPIO nanodots “complex” in an appropriate solvent to dissolve the compound and separate it from the SPIO nanodots by use of a magnet. Generally the superparamagnetic metal nanodots used in this process are coated with a surfactant molecule such as oleic acid to stabilize magnetic nanoparticles through a strong chemical bond between the functional group of the surfactant molecule (e.g., the carboxylic acid of the oleic acid) and the amorphous metal oxide nanoparticles.

The purified compounds from medicinal natural products, or their synthetic analogs and derivatives, are further processed into particulate forms (e.g., microparticles or nanoparticles), optionally encapsulating a therapeutic, prophylactic, or diagnostic agent via emulsion or other techniques. In a preferred embodiment, these compounds form supramolecular nanoparticles via emulsion with a surfactant such as polyvinyl alcohol. In another embodiment, these compounds, generally amphiphilic or hydrophobic, form supramolecular nanoparticles via self-assembly in an aqueous environment.

The isolated and enriched MNP-based compounds, their synthetic analogs and derivatives, and supramolecular particles formed therefrom, provides improved safety besides enhanced drug delivery efficiency, compared with a crude mixture of natural plant/microbial/animal-based product and drug agents for consumption as practiced in some traditional medicines. They are also suitable for administration to a subject via different routes including intravenous administration and local injections.

A chemical extraction approach was used to identify natural materials found in herbs that form NPs. Betulinic acid (BA), a natural compound, was chemically extracted from E. ulmoides, a herb (Tsai, et al. Journal of ethnopharmacology 2017, 200, 31-44; Luo, et al. ACS Chem Neurosci 2014, 5 (9), 855-66). Intravenously administered BA NPs incorporating an antioxidant agent and/or anti-edema agent were shown to penetrate the blood brain barrier and interstitial extracellular matrix barrier into the brain and effectively reduce ischemia-induced infarction. BA NPs enabled efficient delivery of glyburide, an anti-edema agent whose efficacy has been limited by its low brain penetrability, leading to therapeutic benefits significantly greater that those achieved by either glyburide or BA NPs alone. The extraction approach was used to isolate additional nanomaterials which also formed nanoparticles, include ursolic acid (UA), stigmasterol (ST), sumaresinolic acid (SA), glycyrrhetic acid (GA), dehydrotrametenolic acid (DTA), poricoic acid A (PAA), lupeol (LP), β-sitosterol (BT), and oleanolic acid (OA). NPs containing UA, ST, SA, GA, DTA, PAA, LP, BT, or OA effectively promoted stroke recovery after intravenous administration.

At least five classes of MNP-based compounds have been demonstrated to form supramolecular particles for effective delivery of different types of therapeutic, prophylactic, or diagnostic agents. These compounds are isolated from natural sources such as plants. Exemplary MNP-based compounds, from which synthetic analogs or derivatives are made and appreciated to function similarly, e.g., capable of forming supramolecular particles include diterpene resin acids (e.g., abietic acid and pimaric acid), phytosterols (e.g., stigmasterol and β-sitosterol), lupane-type pentacyclic triterpenes (e.g., lupeol and betulinic acid), oleanane-type pentacyclic tritepenes (e.g., glycyrrhetic acid and sumaresinolic acid), and lanostane-type triterpenes and derivatives (e.g., dehydrotrametenolic acid and poricoic acid A).

These compounds are isolated and extracted from natural plant, microbial, or animal products in one or more ways. In a first embodiment, a crude natural product is heated or boiled in water or an aqueous medium in the presence of one or more superparamagnetic metal oxide nanodots (e.g., superparamagnetic iron oxide (SPIO) nanodots), such that compounds capable of forming supramolecular nanoparticles are associated with the superparamagnetic metal nanodots, the complex of which is further isolated using a magnet. In a second embodiment, a plant, microbial, or animal product is immersed in an appropriate organic solvent such as dichloromethane, chloroform, and ethyl acetate, where the dissolved filtrate is collected (i.e., to remove undissolvable impurity and to enrich the compounds for forming supramolecular particles). The organic phase filtrate is emulsified in the presence of one or more superparamagnetic metal nanodots (e.g., SPIO nanodots), such that compounds to form supramolecular particles are associated with the superparamagnetic metal nanodots, forming a “complex” that is further isolated using a magnet. Using either approach, further purification of isolated compounds to separate from the SPIO nanodots usually involves immersing the compound-SPIO nanodots “complex” in an appropriate solvent to dissolve the compound and separate it from the SPIO nanodots by use of a magnet. Generally the superparamagnetic metal nanodots used in this process are coated with a surfactant molecule such as oleic acid to stabilize magnetic nanoparticles through a strong chemical bond between the functional group of the surfactant molecule (e.g., the carboxylic acid of the oleic acid) and the amorphous metal oxide nanoparticles.

The purified compounds from medicinal natural products, or their synthetic analogs and derivatives, are further processed into particulate forms (e.g., microparticles or nanoparticles) encapsulating a therapeutic, prophylactic, or diagnostic agent via emulsion or other techniques. In a preferred embodiment, these compounds form supramolecular nanoparticles via emulsion with a surfactant such as polyvinyl alcohol. In another embodiment, these compounds, generally amphiphilic or hydrophobic, form supramolecular nanoparticles via self-assembly in an aqueous environment.

The isolated and enriched MNP-based compounds, their synthetic analogs and derivatives, and supramolecular particles formed therefrom, provide improved safety besides enhanced agent delivery efficiency, compared with a crude mixture of natural plant/microbial/animal-based product and agents for consumption as practiced in some traditional medicines. They are also suitable for administration to a subject via different routes including intravenous administration, local injections and topical application.

Exemplary classes of MNP-based compounds for supramolecular particles for delivering agents include (i) diterpene compounds; (ii) phytosterols; (iii) lupane pentacyclic triterpenes; (iv) oleanane-type pentacyclic triterpenes; and (v) lanostane-type triterpenes; and compounds similar in structures to compounds in these classes, as well as their derivatives. The classification of compounds are not necessarily mutually exclusive. Compounds in one or two or more classes may be generalized to a broad chemical formula, where individual embodiments form supramolecular particles for enhancing delivery efficiency of agents following administration.

Generally, compounds forming supramolecular particles have a general structure defined by formula 1.

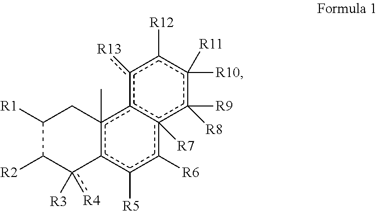

wherein R1 is H, OH, or C(═O)R16; R2 is H or R17; R3 is H, CH₃, or R18; R4, if single bonded, is H, CH₃ or R19, or R4, if double bonded, is CH₂; R5 is H or OH; R6 is H or OH; R7 is H or CH₃; R8 is H or CH₃; R9 is H or R14; R10 is R15 when R9 is R14, or R10 is R20 when R9 is H; R11 is H, CH₃, or R21; R12 is H or OH; R13, if single bonded, is H, or R13, if double bonded, is O or S; R14 and R15 combine to form a five-membered ring, a six-membered ring, or a six-membered ring fused with another five-membered or six-membered ring;

R16, R17, R18, R19, R20, or R21 are individually a derivatizing group comprising an amine, a polyethylene glycol, OH, a carboxyl, an alkyl, an alkene, an amide, a sulphonyl, an aryl, a carbohydrate, or a combination thereof;

wherein each dashed line between two atoms otherwise connected by a solid line indicates, individually, the two atoms are monovalently connected or divalently connected, the number of divalently connection not exceeding allowed valency in fused cyclic rings; and wherein the dash line between two atoms not otherwise connected by a solid line indicates a monovalent bond or no covalent bond.

In some embodiments where R1 is C(═O)R16; R2═R3═R5═R6═R7═R12═H; R13 is single bonded and is H; R4 is double bonded and is CH₂; R8═R11═CH₃; R9 is R14; R10 is R15; R14 and R15 combine to form a five-membered ring; the compounds are defined by formula 2:

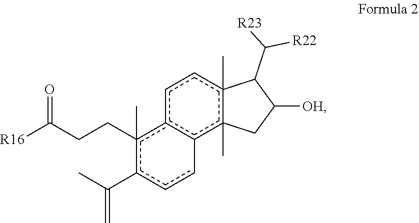

wherein R22 and R23 are individually a derivatizing group comprising a carboxyl, an alkyl, an alkene, a poly(ethylene glycol), an amine, OH, or a combination thereof.

Exemplary compounds having a structure defined by formula 2 include poricoic acid A, poricoic acid AE, derivatives thereof.

In another embodiment where R1═R5═R6═R7═R12═H; R2═OH or R17; R3 is H or CH₃; R4 is H or CH₃; R9 is R14; R10 is R15; R14 and R15 combine to form a five-membered ring; R11 is CH₃; R13 is single bonded and is H; the compounds are defined by Formula 3:

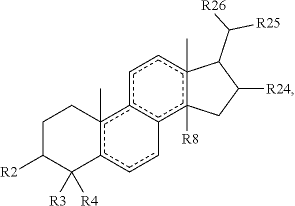

wherein R24 is H or OH; R25 and R26 are individually a derivatizing group comprising a carboxyl, an alkyl, an alkene, a poly(ethylene glycol), an amine, OH, or a carboxyl with the hydrogen replaced by

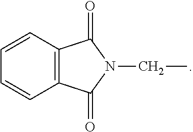

Exemplary compounds defined by formula 3 include dehydrotrametenolic acid, pachymic acid, beta sitosterol, cholesterol, ergosterol, campesterol, stigmasterol, and derivatives thereof.

In yet another embodiment where R1═R3═R4═R5═R7═R8═R13═H; R11 is CH₃; the compounds are defined by formula 4:

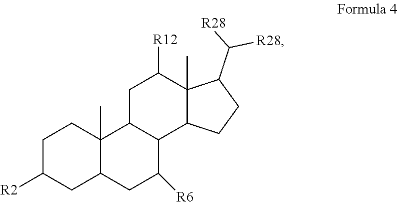

wherein R27 and R28 are individually a derivatizing group comprising a carboxyl, an alkyl, an alkene, a poly(ethylene glycol), an amine, an amide, OH, a sulphonyl.

Exemplary compounds defined by Formula 4 include cholic acid, glycocholic acid, taurocholic acid, deoxycholic acid, lithocholic, glycochenodeoxycholic acid, taurochenodeoxycholic acid, ursodeoxycholic acid, chenodeoxycholic acid, and derivatives thereof.

In yet another embodiment where R1═R2═R5═R6═R7═R8═R9 ═R12═R13═H; the compounds are defined by formula 5:

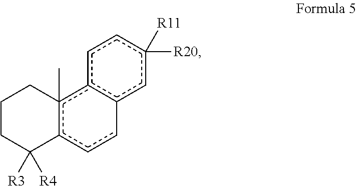

wherein R3, R4, R20 and R11 are individually a derivatizing group comprising a carboxyl, an alkyl, an alkene, a poly(ethylene glycol), an amine, an amide, a sulphonyl, OH, or a combination thereof.

Exemplary compounds defined by formula 5 include isopimaric acid, abietic acid, dehydroabietic acid, isodextropimaric acid, and derivatives thereof.

In yet another embodiment where R1 is H or OH; R4═R7═R8═CH₃; R6═R11═R12═H; R9 is R14; R10 is R15; R14 and R15 combine to form a six-membered ring fused with another five-membered ring; the compounds are defined by Formula 6:

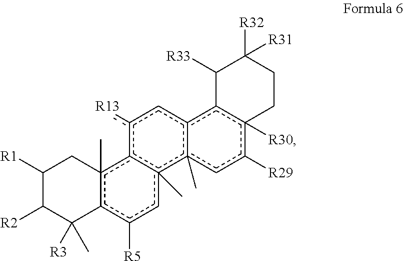

wherein R29 is H or OH; R30, R31, R32, and R33 are individually a derivatizing group comprising a carboxyl, an alkyl, an alkene, a poly(ethylene glycol), an amine, an amide, OH, a sulphonyl, or a combination thereof.

Exemplary compounds defined by Formula 6 are oleanolic acid, ursolic acid, sumaresinolic acid, echinocystic acid, maslinic acid, beta-boswellic acid, glycyrrhetic acid, glycyrrhizic acid, asiatic acid, and derivatives thereof such as these six:

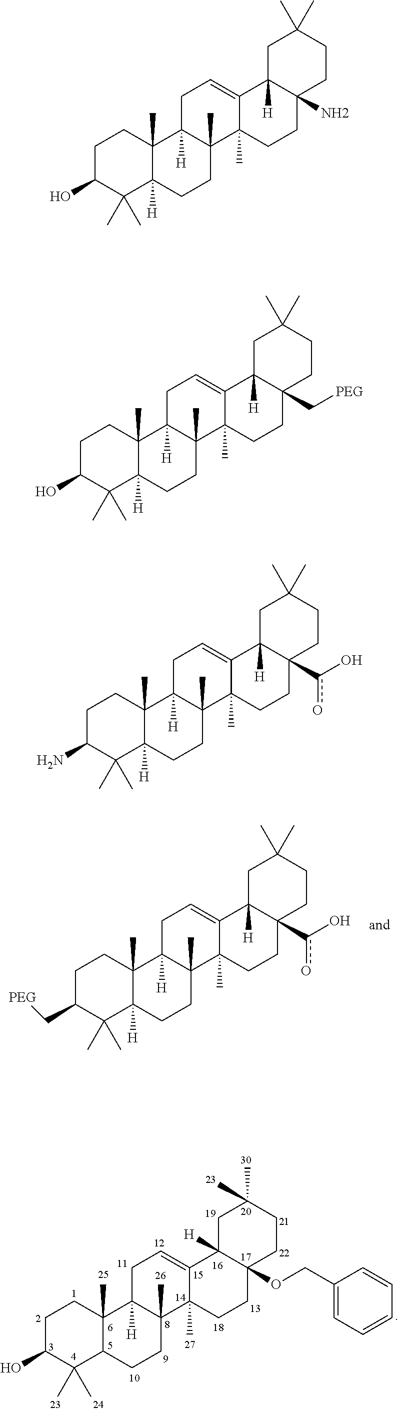

In yet another embodiment where R1═R5═R6═R11═R12═R13 ═H; R7═R8═CH₃; R9 is R14; R10 is R15; R14 and R15 combine to form a six-membered ring fused with another five-membered ring; the compounds defined by formula 7:

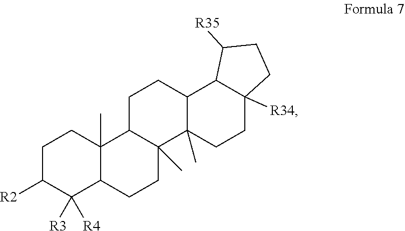

wherein R34 and R35 are individually a derivatizing group comprising a carboxyl, an alkyl, an alkene, a poly(ethylene glycol), an amine, an amide, OH, a sulphonyl, or a combination thereof.

Exemplary compounds defined by Formula 7 include lupeol, betulinic acid, betulin, and derivatives thereof.

These compounds can also be described in the following classes.

### i. Diterpene-Class

Diterpene compounds contain two terpenes, which includes four isoprene units in linear or cyclic forms. Depending on the number of rings of in terpene compounds, there are compounds with no ring such as phytane; with 1 ring such as cembrene A; with 2 rings such as sclarene and labdane; with three rings such as abietane and taxadiene; and with 4 rings such as stemarene and stemodene.

Exemplary diterpene compounds include abietic acid, dehydroabietic acid, pimaric acid, isopimaric acid, and isodextropimaric acid with the following formulae.

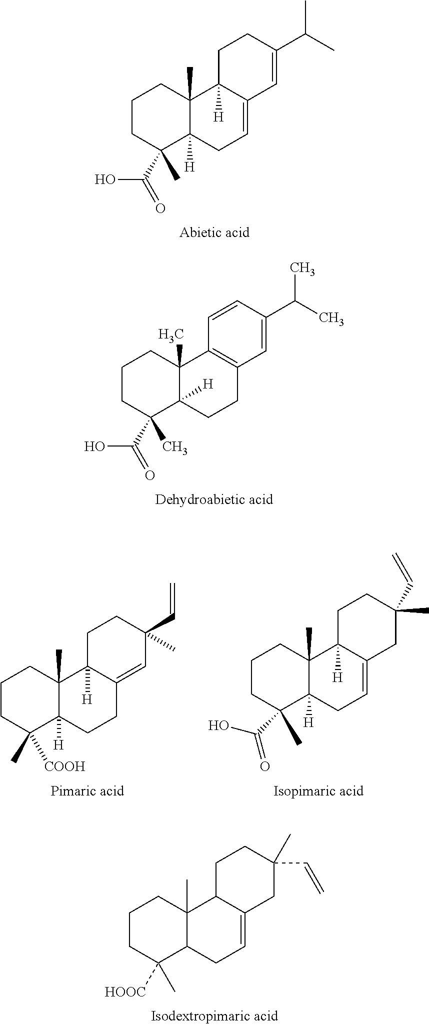

### ii. Phytosterol-Class or Phytosterol-Like

Phytosterols are capable of forming supramolecular particles with heating and/or dissolution in appropriate solvent for encapsulation of. Exemplary phytosterols include stigmasterol, ergosterol, beta sitosterol, cholesterol campesterol with the following formula.

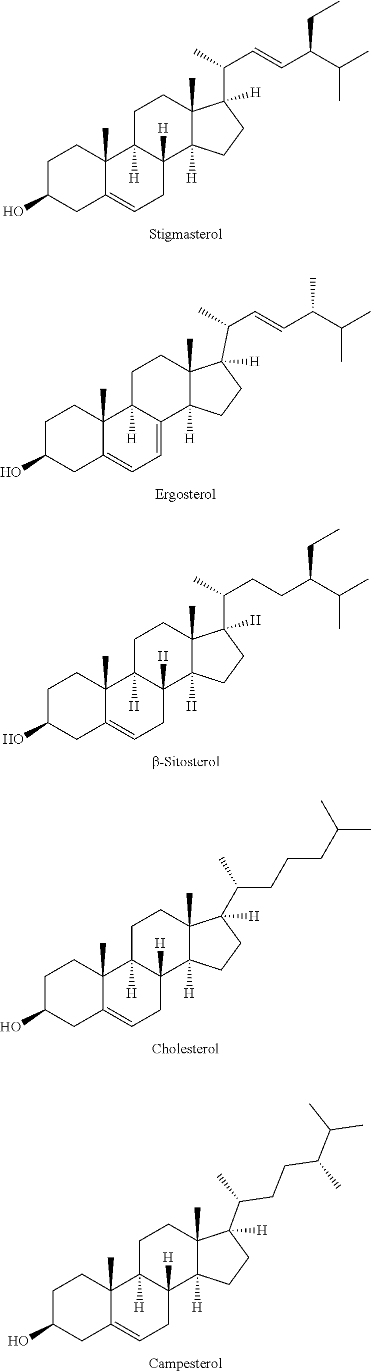

Although phytosterols may be isolated from botanical, microbial, and/or animal natural products, it is appreciated by one skilled in the art the synthetic variant and its derivatives will include similar properties to encapsulate agents based on the disclosure in this application.

### iii. Lupane Pentacyclic Triterpenes

Lupane pentacyclic triterpenes are capable of forming nanoparticles with heating and/or dissolution in appropriate solvent for encapsulation of agents. Exemplary lupane pentacyclic triterpene include lupeol, betulinic acid, and betulin with the following formulae.

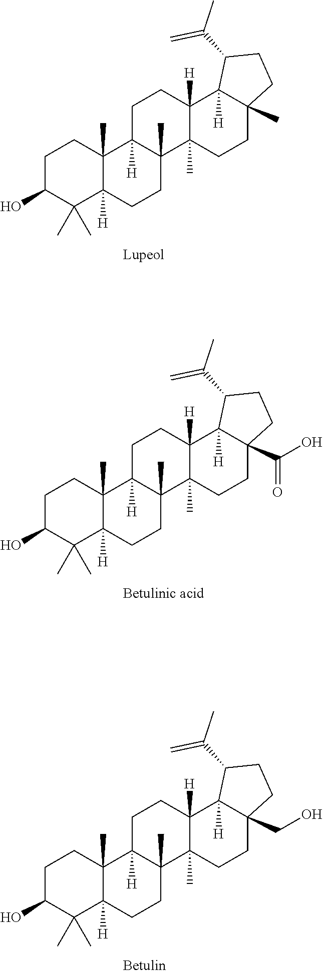

Although pentacyclic triterpenes may be isolated from botanical, microbial, and/or animal natural products, it is appreciated by one skilled in the art the synthetic variant and its derivatives will include similar properties to encapsulate agents for high efficiency agent delivery based on the disclosure in this application.

### iv. Oleanane Type Triterpenes or Pentacyclic Triterpenoids

Pentacyclic triterpenes or pentacyclic triterpenoid-based compounds are capable of forming nanoparticles with heating and/or dissolution in appropriate solvent for encapsulation of agents. Exemplary pentacyclic triterpene or triterpenoid-based compound include sumaresinolic acid, glycyrrhetic acid, oleanolic acid, ursolic acid, echinocystic acid, maslinic acid, β-boswellic acid, and glycyrrhizic acid with the following formulae.

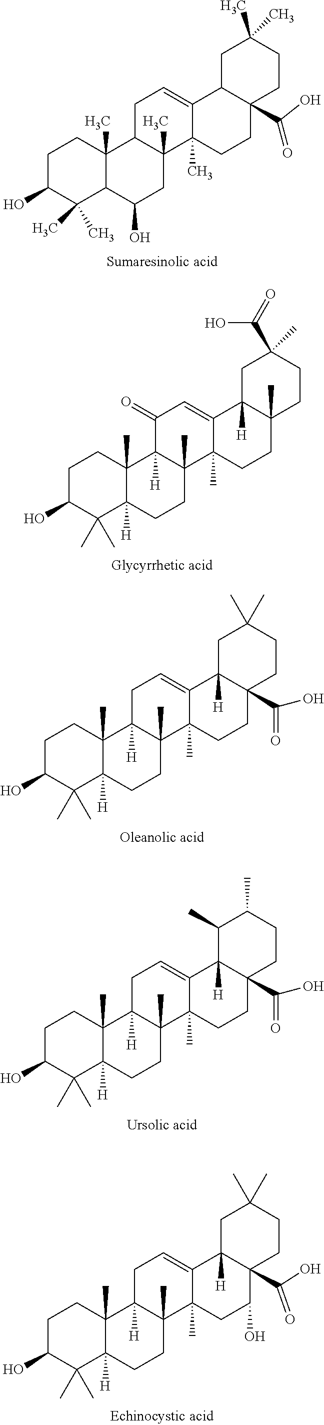

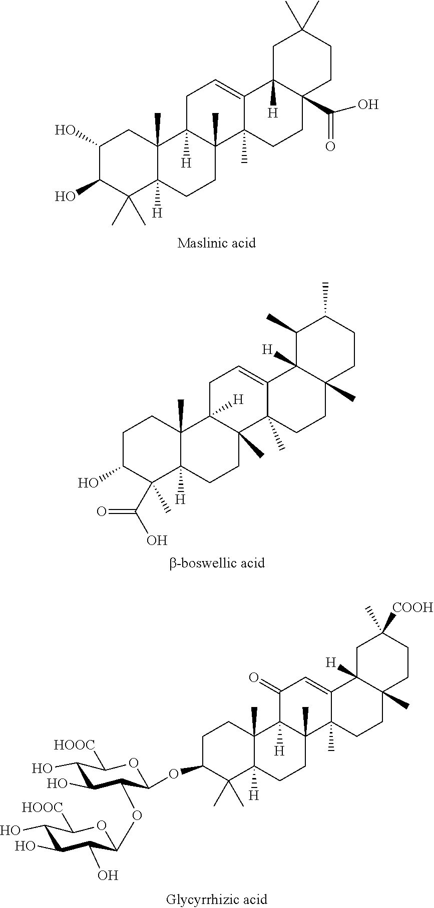

### v. Lanostane-Type Triterpenes and Derivatives

Triterpene compounds contain three terpenes, which includes six isoprene units in linear or cyclic forms. Tetracyclic triterpene-based compounds are capable of forming nanoparticles with heating and/or dissolution in appropriate solvent for encapsulation of agents.

Exemplary tetracyclic triterpene compounds include dehydrotrametenolic acid, trametenolic acid, poricoic acid A, poricoic acid B, poricoic acid AE with the following formulae.

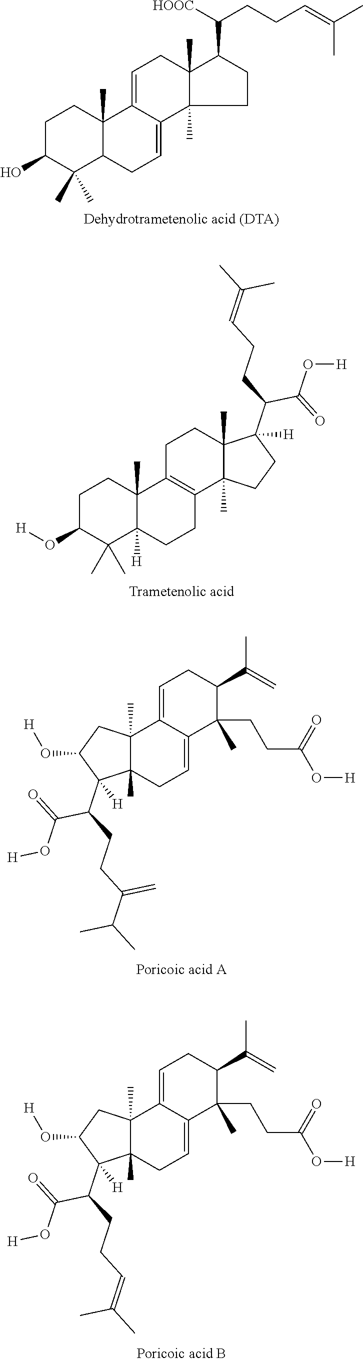

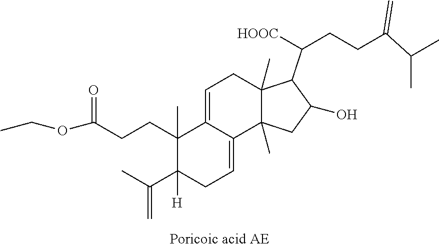 [SMILES: C=C(CCC(C(=O)O)C1C(O)CC2(C)C3=CCC(C(=C)C)C(C)(CCC(=O)OCC)C3=CCC12C)C(C)C]

Tetracyclic triterpene derivatives capable of forming nanoparticulate morphology for encapsulation of agents include those derived from substitution at one or more positions, e.g., by alkyl, alkylene, alkenyl, alkynyl, alkoxy, alkylamino, alkylthio, carbonyl, carboxyl, amido, sulfonyl, sulfonic acid, sulfamoyl, sulfoxide, phosphoryl, or phosphonyl of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbons. Although tetracyclic triterpene compounds may be isolated from botanical, microbial, and/or animal natural products, it is appreciated by one skilled in the art that synthetic variant and its derivatives will include similar properties to encapsulate agents.

2. Morphology and Properties of Formed Supramolecular Particles

MNPs-based compounds, their synthetic analogs or derivatives and agents to be delivered are dissolved in appropriate solvent (e.g., organic solvent such as dichloromethane, chloroform, ethyl acetate) where these compounds form supramolecular particles via non-covalent interactions that encapsulate, associate, or otherwise incorporate agents to be delivered. Inclusion of a surfactant may further improve the morphology of the formed supramolecular particles and reduce aggregation.

In one embodiment where boiling and cooling are used to extract/purify compounds from plants and other natural product, exemplary heating temperature includes about 40, 50, 60, 70, 80, 90, 100, and 110° C. Exemplary cooling temperature includes about 30, 25, 20, 15, 10, 5, and 0° C.

In some embodiments, the MNP-based compounds, their synthetic analogs or derivatives are emulsified in the presence of a surfactant to form supramolecular particles via non-covalent associations. Exemplary surfactants in forming supramolecular particles include anionic, cationic and non-ionic surfactants, such as, but not limited to, polyvinyl alcohol, F-127, lectin, fatty acids, phospholipids, polyoxyethylene sorbitan fatty acid derivatives, and castor oil. Other suitable surfactants include L-α-phosphatidylcholine (PC), 1,2-dipalmitoylphosphatidylcholine (DPPC), oleic acid, sorbitan trioleate, sorbitan mono-oleate, sorbitan monolaurate, polyoxyethylene (20) sorbitan monolaurate, polyoxyethylene (20) sorbitan monooleate, natural lecithin, oleyl polyoxyethylene (2) ether, stearyl polyoxyethylene (2) ether, lauryl polyoxyethylene (4) ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, and sunflower seed oil, lecithin, oleic acid, and sorbitan trioleate.

Agent-containing supramolecular particles may be microparticles or nanoparticles of any shape. In some embodiments, supramolecular nanoparticles have a spherical or about spherical shape with an average diameter ranging from 10 nm and 700 nm, preferably between 50 nm and 500 nm, more preferably between 50 nm and 200 nm. They may also be in the form of nanorods with an average length ranging from 50 nm to 800 nm, preferably between 300 nm and 500 nm, with an average width between 5 nm and 180 nm, most preferably between 10 nm and 50 nm. Techniques to observe and measure nanostructures include scanning electron microscopy, transmission electron microscopy, atomic force microscopy, and/or dynamic light scattering. Particles of other geometries and sizes (e.g., microparticles) may be prepared from the MNP-based compounds.

The supramolecular particles may encapsulate therapeutic agents that are hydrophilic or hydrophobic.

These nanoparticles generally have a negative surface charge, e.g., having zeta-potential at physiological environment between about 0 mV and −50 mV, or between −10 mV and −30 mV. They are generally acid stable, e.g., do not break or deform and excessively leak encapsulated agent in an acidic environment.

3. Solvent

Suitable organic solvents to extract and purify from medicinal natural products the one or more compounds capable of forming supramolecular particles include, but are not limited to, a polar or non-polar solvent, such as dichloromethane, DMSO, dipropylene glycol, propylene glycol, hexyl butyrate, glycerol, acetone, dimethylformamide (DMF), tetrahydrofuran, dioxane, acetonitrile, alcohol (e.g., ethanol, methanol or isopropyl alcohol, butyl alcohol, pentyl alcohol), benzene, toluene, carbon tetrachloride, acetonitrile, glycerol, 1,4-dioxane, dimethyl sulfoxide, ethylene glycol, chloroform, hexane, tetrahydrofuran, xylene, mesitylene, and/or any combination thereof. An organic solvent is generally selected based on the solubility of the crude and fine medicinal natural products therein, and may be affected by the polarity, hydrophobicity, water-miscibility, and in some cases the acidity of the solvent. Preferred solvents are those regarded by the U.S. Food and Drug Administration as “GRAS” (“generally regarded as safe”).

MNP-based compounds for encapsulation of agents in a supramolecular particle form are typically purified from the extracts of different plant species such as Poria cocos, Artemisia annua L, Taxus, and Radix Glycyrrhizae. One or more approaches may be used to isolate and purify these compounds, including aqueous boiling and chemical (organic solvent) extraction methods with the help of superparamagnetic nanoparticles. Purification method generally achieves about 100%, 95%, 90%, 85%, 80%, 75%, or 70% purity of the MNP compounds capable to form supramolecular particles, as measured by techniques such as high performance liquid chromatography or mass spectrometry.

Isolated compounds, especially via chemical extraction method, are generally purified to remove the organic solvent. Column chromatography, drying in vacuo, lyophilization, filtration, and centrifugation are exemplary techniques to separate the MNP-based compounds from solvents or impurities.

B. Therapeutic, Prophylactic and Diagnostic Agents

The supramolecular particles may contain one or more therapeutic, prophylactic, and/or diagnostic agents, jointly referred to herein as “agents”. Therapeutic, prophylactic and diagnostic agents may be proteins or peptides, sugars or polysaccharides, lipids, lipoproteins or lipopolysaccharides, nucleic acids (DNA, RNA, siRNA, miRNA, tRNA, piRNA, etc.) or analogs thereof, or small molecules (organic, inorganic, natural or synthetic). In some embodiments, the nucleic acid is an expression vector encoding a protein or a functional nucleic acid. Vectors can be suitable for integration into a cell genome or expressed extra-chromosomally. In other embodiments, the nucleic acid is a functional nucleic acid. Suitable small molecule active agents include organic and organometallic compounds. The small molecule active agents can be hydrophilic, hydrophobic, or amphiphilic compounds.

Exemplary therapeutic or prophylactic agents include, but are not limited to, chemotherapeutic agents, neurological agents, tumor antigens, CD4+ T-cell epitopes, cytokines, small molecule signal transduction inhibitors, photothermal antennas, immunologic danger signaling molecules, other immunotherapeutics, enzymes, antimicrobials or antivirals, anti-parasitics, growth factors or inhibitors, hormones or hormone antagonists, antibodies and bioactive fragments thereof (including humanized, single chain, and chimeric antibodies), antigen or vaccine formulations (including adjuvants), anti-inflammatories or immunomodulators (including ligands that bind to Toll-Like Receptors, including, but not limited to, CpG oligonucleotides) to activate the innate immune system, molecules that mobilize and optimize the adaptive immune system, molecules that activate or up-regulate the action of cytotoxic T lymphocytes, natural killer cells and helper T-cells, and molecules that deactivate or down-regulate suppressor or regulatory T-cells), agents that promote uptake of the nanoparticles into cells (including dendritic cells and other antigen-presenting cells), oligonucleotide drugs (including DNA, RNAs, antisense, aptamers, small interfering RNAs, ribozymes, external guide sequences for ribonuclease P, and triplex forming agents) and other gene modifying agents such as ribozymes, CRISPR/Cas, zinc finger nuclease, and transcription activator-like effector nucleases (TALEN).

Exemplary diagnostic agents include paramagnetic molecules, fluorescent compounds, magnetic molecules, radionuclides, x-ray imaging agents, and contrast agents.

The MNPs enhance bioavailability following administration and/or improve targeting and therapeutic efficacy. The MNP molecules form supramolecular particles through noncovalent interactions (also termed functional nanomaterials or micromaterials). The supramolecular particles can form based on hydrogen-bonding interactions, π-π interactions, solvophobic-solvophobic interactions, a combination thereof, or other non-covalent intermolecular interactions among the MNP-based compounds. In some embodiments, the structures of these molecules in formed supramolecular particles are planar or near planar with a stack or slipped-stack geometry. Any encapsulated agents in these supramolecular particles are efficiently transported and delivered. Alternatively, agents may be associated or bonded with these compounds; or they may be entrapped, non-covalently associated, or covalently bonded within, or on, the surface of, nanoparticles formed from these MNP-based compounds.

Compared with delivering unencapsulated agent, the supramolecular particles exhibit a greatly improved efficiency in preferential accumulation in different tissues including the brain.

A wide variety of agents can be encapsulated, associated, bonded, or otherwise carried by supramolecular particles formed via noncovalent association of these enriched MNP-based compounds, their synthetic analogs and derivatives for treatment of different diseases and disorders. The isolated MNP-based small molecule compounds, generally amphiphilic or hydrophobic, are more enriched and purified compared to their original form in MNP. For example, the purity of such compounds after isolation and enrichment from MNP increases to greater than 80%, 85%, 90%, 95%, 97%, 98%, or 99% by weight. Examples include extracted poricoic acid A (PAA) and dehydrotrametenolic acid (DTA) from Poria cocos form supramolecular nanoparticles.

In one embodiment, the preferred agent targets a pathological processes of stroke, such as cerebral edema, oxidative stress, excitotoxicity, and inflammation. A preferred example is glyburide, an antagonist to the SUR1-TRPM4 cation channel that targets cerebral edema (Simard, et al., Nature medicine 2006, 12 (4), 433-40; Sheth, et al. Lancet Neurol 2016, 15 (11), 1160-1169).

Herbal medicine has been widely used for clinical management of various diseases in human history, as reported by Farnsworth, et al. Bulletin of the World Health Organization 1985, 63 (6), 965-81. A recent analysis suggests that over 200 medicinal herbs might be effective on stroke. Zhang, et al. J. traditional and complementary medicine 2014, 4 (2), 77-81; Feigin, Stroke; a journal of cerebral circulation 2007, 38 (6), 1734-6; Liu, et al. Sci Rep 2017, 7, 41406.

Although medicinal natural products (MNPs) accounted for more than half of the newly developed small molecule drugs over the period 1981-2010 (Newman, D. J. & Cragg, G. M., J Nat Prod 75, 311-335 (2012)), a major obstacle to MNP-based drug discovery is that over 90% of the isolated compounds cannot be used as drugs because of their poor stability, solubility, or pharmacokinetics. As a result, chemical alterations or specific formulations of MNPs are often required for clinical applications (Kumari, A., et al., Trends in Medical Research 7, 34-42, (2012); Sucher, N J., Epilepsy Behav 8, 350-362, (2006)). Exemplary compounds include artemisinin (Balint G A, et al., Pharmacology & therapeutics, 90, 261-265 (2001)), paclitaxel (P T X, Singla A K, et al, Int J Pharm, 235, 179-192 (2002)), and curcumin (Anand P, et al., Mol Pharm, 4, 807-818 (2007)). Artemisinin, a compound purified from Artemisia annua L, has a bioavailability of less than 10% and is used mostly in its derivative forms (Balint, G. A., et al., Pharmacology & therapeutics 90, 261-265 (2001)). Paclitaxel, a compound purified from Taxus species, has poor solubility in aqueous solution and needs to be formulated, for example, with Cremophor EL for clinical applications (Singla, A. K., et al., Int J Pharm 235, 179-192 (2002)). Additionally, some MNPs such as Poria cocos, although commonly used in traditional medicine, do not contain pharmacologically active components. Some MNPs such as Radix Glycyrrhizae and glycyrrhizin when co-administered may enhance the bioavailability of certain pharmaceutically active drugs, although these MNP extracts do not appear to contain active components (Kesarwani, K., et al., Asian Pac J Trop Biomed 3, 253-266 (2013); Fasinu, P. S., et al., Frontiers in pharmacology 3, 69 (2012)). Most compounds isolated from herbs are known to have a limited ability to penetrate the brain (Fricker Curr Drug Metab 2008, 9 (10), 1019-1026).

The therapeutic agent can also be or include one or more neuroprotective agents. In general, neuroprotective agents are medications that can alter the course of metabolic events after the onset of a brain injury, such as ischemia. Preferably, the neuroprotective agent can prevent damage to the brain from ischemia, stroke, convulsions, or trauma. Some neuroprotective agents must be administered before the event, but others may be effective for some time after. The neuroprotective agent can act by a variety of mechanisms, but often directly or indirectly minimize the damage produced by endogenous excitatory amino acids. Exemplary neuroprotective agents include Tat-NR2B9c (also referred to as “NA1” peptide) and the poly-arginine R18 peptide. Other neuroprotective agents, such as Tat-NR2B9c, can be used for treating strokes.

Preferred therapeutic agents target various pathological processes of acute brain injuries, such as cerebral edema (such as glyburide), oxidative stress (such as butylphthalide), excitotoxicity (such as NA1), inflammation (such as fingolimod), and platelet aggradation (such as ticagrelor).

Examples of other agents that may be included in the formulations for these applications include glyburide, Tat-NR289c (also called NA-1), minocycline, S1P agonists like fingolimod/saponimod, uric acid, IL-6 receptor antagonists, Factor XII inhibitors, 3K3A-APC, rock inhibitors, avastin, vegf-trap, NEP1-40.

The particles can also be targeted to specific tissues or sites of injury. Examples of ligands to targeting to stroke and ischemia include targeting ligands include: AMD31000 (a ligand for CXCR4), chlorotoxin (CTX), anti-TfR antibody, and anti-fibrin antibody.

Betulinic acid (BA), a natural compound that forms nanoparticles (NPs) was chemically extracted from E. ulmoides, a herb (Tsai, et al. Journal of ethnopharmacology 2017, 200, 31-44; Luo, et al. ACS Chem Neurosci 2014, 5 (9), 855-66. BA NPs were capable of efficiently penetrating ischemic brains and effectively promoting functional recovery as antioxidant agents in animal models where stroke was induced by middle cerebral artery occlusion (MCAO). BA NPs significantly enhances the delivery of a therapeutic agent such as glyburide, which has a limited ability to penetrate the ischemic brain as determined by positron emission tomography-computed tomography (PET/CT), resulting in therapeutic benefits greater than those achieved by either glyburide or BA NPs alone.

Additional materials identified using the same approach which also formed nanoparticles, include ursolic acid (UA), stigmasterol (ST), and oleanolic acid (OA). NPs containing UA, ST, or OA effectively promoted stroke recovery after intravenous administration. Based on the discussion above, one skilled in the art could identify other useful compounds having the requisite backbones to make them form NPs. R groups could vary.

The amount of agent to be encapsulated in the supramolecular particles depends on the molecular weight, hydrophobicity/hydrophilicity, and polarity of the agent to be encapsulated and that of the supramolecular particle-forming compounds. Generally, agents to be delivered are prepared with MNP-based compounds, their synthetic analogs or derivatives, at between about 1% and 80% by weight, preferably between about 5% and 70% by weight. Agent encapsulation efficiency may be about 100, 90, 85, 80, 70, 60, or 50%, with a agent loading efficiency in the formed nanoparticles of about 5, 7.5, 10, 15, 20, 30, 40, or 50%. Agent loading represents the weight content of agent in supramolecular particles. Agent encapsulation efficiency represents the ratio of final agent loading in comparison to the theoretical agent loading.

In some forms, the nanoparticles without agent to be delivered (also referred to as “empty nanoparticles”) exhibit therapeutic effect and can also be used therapeutically, for example, to treat stoke.

C. Formulations and Excipients

The formulations are designed for distribution and storage or for administration. For example, the NPs may be in lyophilized or powder form in a single dosage unit container into which diluent/suspending fluid is added at the time of administration. These may be distributed in dosage unit form containing an amount for treatment of a particular disease or disorder, size of patient and/or via a particular route of administration. These may also be distributed in combination with a diluent/resuspending agent.

Formulations may be defined as are prepared using a pharmaceutically acceptable “carrier” composed of materials that are considered safe and effective and may be administered to an individual without causing undesirable biological side effects or unwanted interactions. Standard textbooks for formulating include “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et. al., (Media, P A: Williams and Wilkins, 1995).

The NPs are typically administered by injection (intravenous, intramuscular, subcutaneous), or may be administered topically to a mucosal tissue (nasal, buccal, pulmonary, vaginal, rectal). In the preferred embodiment, the NPs are administered by injection, typically in an aqueous vehicle. “Parenteral administration”, as used herein, means administration by any method other than through the digestive tract or non-invasive topical or regional routes. For example, parenteral administration may include administration to a patient intravenously, intradermally, intraperitoneally, intrapleurally, intratracheally, intramuscularly, subcutaneously, subjunctivally, by injection, and by infusion.

Pharmaceutical formulations for parenteral administration are preferably in the form of a sterile aqueous solution or suspension of particles formed from one or more polymer-agent conjugates. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol.

Pulmonary administration”, as used herein, means administration into the lungs by inhalation or endotracheal administration. As used herein, the term “inhalation” refers to intake of air to the alveoli. The intake of air can occur through the mouth or nose.

Suitable excipients for formulating NPs for these routes of administration are known. In some instances, the formulation is distributed or packaged in a liquid form. Alternatively, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration may be buffered with an effective amount of buffer necessary to maintain a pH suitable for administration. Suitable buffers are well known by those skilled in the art and some examples of useful buffers are acetate, borate, carbonate, citrate, and phosphate buffers. Solutions, suspensions, or emulsions for parenteral administration may also contain one or more tonicity agents to adjust the isotonic range of the formulation. Suitable tonicity agents are well known in the art. Examples include glycerin, mannitol, sorbitol, sodium chloride, and other electrolytes.

Solutions, suspensions, or emulsions for parenteral administration may also contain one or more preservatives to prevent bacterial. Suitable preservatives are known in the art, and include polyhexamethylenebiguanidine (PHMB), benzalkonium chloride (BAK), stabilized oxychloro complexes (otherwise known as PURITE®), phenylmercuric acetate, chlorobutanol, sorbic acid, chlorhexidine, benzyl alcohol, parabens, thimerosal, and mixtures thereof.

Solutions, suspensions, or emulsions for parenteral administration may also contain one or more excipients known art, such as dispersing agents, wetting agents, and suspending agents.

Aerosols for the delivery of therapeutic agents to the respiratory tract have been described, for example, Adjei, A. and Garren, J. Pharm. Res., 7: 565-569 (1990); and Zanen, P. and Lamm, J. W. J. Int. J. Pharm., 114: 111-115 (1995). Gonda, I. “Aerosols for delivery of therapeutic and diagnostic agents to the respiratory tract,” in Critical Reviews in Therapeutic Drug Carrier Systems, 6:273-313 (1990). The deep lung, or alveoli, are the primary target of inhaled therapeutic aerosols for systemic agent delivery. Inhaled aerosols have been used for the treatment of local lung disorders including asthma and cystic. Dry powder formulations (“DPFs”) with large particle size have improved flowability characteristics, such as less aggregation (Visser, J., Powder Technology 58: 1-10 (1989)), easier aerosolization, and potentially less phagocytosis. Dry powder aerosols for inhalation therapy are generally produced with mean diameters primarily in the range of less than 5 μm

### III. Methods of Making

Methods for the preparation of the MNPs and formulations thereof are described in the examples. It is understood that the methods and materials are generally applicable and not limited to the specific examples.

A. Isolation of MNP-Based Compounds Capable of Forming Nanoparticles

Chemical Extraction Method

The MNP source is dissolved in an appropriate solvent, e.g., organic solvent such as dichloromethane, and subsequently emulsified with superparamagnetic metal oxide nanoparticles (e.g., nanodots), resulting in MNP-based compounds associated with the magnetic nanomaterials. The MNP are isolated by applying a magnetic force.

The supramolecular particles-forming MNP-based compounds are separated from the magnetic nanomaterials by dissolving them in an appropriate solvent. Subsequent workup includes washing away/diluting the surfactant, and removing the magnetic nanomaterials by applying a magnetic force.

Suitable superparamagnetic nanoparticles for isolation of compounds from the MNP source include superparamagnetic iron oxide (FeOx, e.g., Fe₃O₄) nanodots or nanocolloids, cobalt nanodots, semi-conducting metals such as Ga, Mn, As, Pt. One or more stabilizing agents or surfactants may coat the surface of these superparamagnetic nanoparticles including oleic acid or sodium oleate. Superparamagnetism (SPM) is a type of magnetism that occurs in small ferromagnetic or ferrimagnetic nanoparticles. This implies sizes around a few nanometers to a couple of tenth of nanometers, depending on the material. Additionally, these nanoparticles are single-domain particles.

Boiling/Soup Method

A MNP source can be boiled in water or an aqueous environment for 30 minutes, one hour, two hours, three hours, or longer. After cooling to room temperature, the MNP can be collected by centrifugation and frozen and/or lyophilized for analysis of supramolecular particle structures under electron microscopy. After cooling, it can also be extracted via the chemical extraction method as described above.

B. Preparing Supramolecular Particles

The MNP-based compounds, their synthetic analogs or derivatives, self-assemble into supramolecular particles via non-covalent interactions. One or more therapeutic, prophylactic, or diagnostic agents are encapsulated or otherwise associated with the self-assembled particles, generally nanoparticles in the spherical shape or the rod shape.

Alternatively, the MNP-based compounds, their synthetic analogs or derivatives are processed into supramolecular particles to encapsulate or otherwise associate with one or more agents. Techniques for making particles include, but are not limited to, emulsion, solvent evaporation, solvent removal, spray drying, phase inversion, low temperature casting, and nanoprecipitation. The therapeutic, prophylactic, or diagnostic agent and pharmaceutically acceptable excipients, including pH modifying agents, disintegrants, preservatives, and antioxidants, can optionally be incorporated into the particles during particle formation. As described above, one or more additional active agents can also be incorporated into the nanoparticle during particle formation.

The preferred method to make the nanoparticles is emulsion. In this method, the MNP-based compounds, their synthetic analogs or derivatives are dissolved in a volatile organic solvent, such as methylene chloride. The organic solution containing the MNP-based compounds, their synthetic analogs or derivatives is then suspended in an aqueous solution that contains a surface active agent such as poly(vinyl alcohol). The agents depending on the solubility may be dissolved in the organic solution or the aqueous solution. The resulting emulsion is stirred until most of the organic solvent evaporated, leaving solid nanoparticles. The resulting particles are washed with water and dried in a lyophilizer or in vacuo. Supramolecular particles with different sizes and morphologies can be obtained by this method. Single emulsion (e.g., oil-in-water) and double emulsion (e.g., water-in-oil-in water) are both suitable for forming supramolecular particles.

### IV. Methods of Treating

The MNPs can be used to treat a variety of diseases and disorders.

Stroke is a leading cause of mortality and morbidity worldwide. There are two main types of stroke, ischemic and hemorrhagic stroke, with the former one accounting for about 87% of all cases. Despite the high prevalence, there are no effective pharmacotherapies targeting brain tissues for stroke. Intravenous tissue-type plasminogen activator (tPA) administered within three hours of symptom onset is the only FDA-approved therapeutic for clinical management of stroke, which functions by dissolving the clots in blocked blood vessels.

Two factors complicate the development of pharmacological therapies for stroke treatment. First, the brain possesses the blood-brain barrier (BBB), which prevents the penetration of most agents to the brain. The BBB is partially disrupted after ischemic insult. However, the degree of disruption may not be sufficient to allow delivery of pharmacologically significant quantities of drugs for effective treatment. Second, there is a lack of effective therapeutic regimens.

In summary, stroke is a major disease without effective pharmacotherapies. The lack of pharmacotherapies can be attributed to two major reasons. First, most therapeutic agents cannot efficiently penetrate the brain because of the existence of the blood brain barrier (BBB). Second, accumulating evidence suggests that single agent pharmacotherapy may be insufficient and effective treatment of stroke requires targeting multiple complementary targets.

The formulations described herein are useful for the treatment of stroke and other ischemic injuries, as well as injuries resulting from traumatic brain injury and the side effects of brain tumors and treatment with surgery and chemotherapy.

The specific dosages and dosing schedules will be determined based on the agent being delivered, its pharmacokinetics in these nanoparticles, the route of administration, the timing of the injury to the brain or ischemic tissue, patient size, and response to treatment.

The present invention will be further understood by reference to the following non-limiting examples.

The formulations can be administered to a subject via different routes including intravenous injections and local injections.

### Example 1: Penetration into the Brain of Glyburide

Glyburide has a limited ability to penetrate the BBB and intravenous administration of glyburide cannot achieve a therapeutic level in the brain (Tournier, et al. Aaps J 2013, 15 (4), 1082-90; Lahmann, et al. PloS one 2015, 10 (7), e0134476). This may be due to inadequate delivery of glyburide to the ischemic brain.

Substantial evidence suggests that SUR1, the molecular target of glyburide, is highly expressed in cells in the neurovascular unit, including neurons, astrocytes, and oligodendrocytes after stroke, which contribute significantly to cerebral edema (Simard, et al. Nature medicine 2006, 12 (4), 433-40 22; Kahle, et al Physiology (Bethesda) 2009, 24, 257-65; Liang, et al. Neurosurgical focus 2007, 22 (5), E2; Sheth, Stroke; a journal of cerebral circulation 2013, 44 (6 Suppl 1), S136). It is clear that further improving the efficacy of glyburide requires enhancing the delivery of glyburide beyond the BBB to allow its engagement with neurovascular cells.

### Materials and Methods

Animals

Male Wistar rat (Charles River Laboratories), ˜200 g each, were given free access to food and water before all experiments. All animal experiments were approved by the Yale University Institutional Animal Care and Utilization Committee.

Middle Cerebral Artery Occlusion (MCAO) Model

MCAO models were generated according to Cai, et al. Neurosci Lett 2015, 597, 127-31; Guo, et al. ACS nano 2018, 12 (8), 8723-8732; Han, et al. Nanomedicine 2016, 12 (7), 1833-42; Yu, et al. Advanced Materials 2018, 30, 1705383. Briefly, rats were anesthetized with 5% isoflurane (Aerrane, Baxter, Deerfield, Ill.) in 30% O₂/70% N₂O using a Tabletop Anesthesia system (Harvard Apparatus, USA). Isoflurane was then maintained at 1.5%. During the procedures, the body temperature of mice was maintained at 37.0±0.5° C. Regional cerebral blood flow (rCBF) was monitored using a laser Doppler flowmeter (AD Instruments Inc.) duration the course of surgery. Mice were placed in the supine position, and a middle neck incision was made under a dissecting microscope (Leica A60). The right common carotid artery (CCA), external carotid artery (ECA), and internal carotid artery (ICA) were carefully exposed and dissected from the surrounding tissue. Then, a small hole in the ECA was made using Vanes-style spring scissors. A 4-0 silicon-coated mono-filament suture (Ducal Corporation) was introduced into the ECA and gently advanced from the lumen of the ECA into the ICA at a distance of 18-20 mm beyond the bifurcation to occlude the origin of middle cerebral artery. Successful MCA occlusion was confirmed by a reduction of rCBF by over 80%. The occlusion lasted 6 hours and the monofilament was withdrawn to allow for reperfusion.

TTC Staining

After euthanization, the brains were isolated, frozen at −20° C. for 30 min, and sliced into 6 coronal slices (2 mm thick). The brain slices were then incubated with 2% TTC in PBS solution at 37° C. for 15 min and fixed in 4% paraformaldehyde.

### Synthesis of ¹¹C-Labeled Glyburide

[¹¹C]Glyburide was synthesized by [¹¹C]-methylation of its desmethyl precursor with [¹¹C]MeOTf in a TRACERLab™ FxC automated synthesis module (GE Medical Systems). [¹¹C]CO₂ was produced via the ¹⁴N(p,α)¹¹C reaction in a PETtrace cyclotron (GE, Milwaukee, Wis.) by bombardment of a target filled with 1% oxygen in nitrogen. [¹¹C]CO₂ was the reacted with hydrogen at 400° C. under a nickel catalyst to afford [¹¹C]CH₄, which was converted to [¹¹C]CH₃I by a gas phase reaction with iodine. [¹¹C]CH₃I was then swept through the silver triflate column at 190° C. and the resulting [¹¹C]CH₃OTf was bubbled into the solution of desmethyl glyburide (1.0 mg) in acetone (0.4 mL) and 3 N NaOH (8 μL) cooled at −10° C. until activity peaked. The reaction mixture was heated at 110° C. for 5 min, cooled to room temperature, diluted with 1.0 mL of 0.1% trifluoroacetic acid (TFA) and injected onto the semi-preparative HPLC column (Luna C18(2), 10 μm, 10×250 mm). The column was eluted with a mobile phase of 55% MeCN and 45% 0.1 M TFA solution at a flow rate of 5 mL/min. The radioactivity fraction eluting between 10-11 min was collected, diluted with a solution of 300 mg of United States Pharmacopeia (USP) grade ascorbic acid in 40 mL of deionized (DI) water, and then loaded onto a Waters Classic C18 SepPak cartridge. The SepPak was rinsed with a solution of 10 mg USP ascorbic acid in 10 mL of DI water, and dried with air. The product was eluted off the SepPak with 1 mL of USP absolute ethanol (Pharmco-AAPER) followed by a solution of 3 mg USP ascorbic acid in 3 mL of USP saline (American Regent). The resulting solution was passed through a sterile 0.22 μm membrane filter (33 mm, MILLEX® GV, Millipore) into a sterile vial pre-charged with 7 mg of USP ascorbic acid in 7 mL of USP saline.

Radiochemical purity and molar activity of [¹¹C]glyburide was determined by HPLC analysis using an Shimadzu Prominence system equipped with a LC-20AT pump, a Luna C18 column (5 μm, 4.6 mm×250 mm), and a SPD-20A UV/Vis detector connected in series with a Bioscan Flow-Count gamma-detector. The system was eluted with a mobile phase of 53% CH₃CN with 47% of 0.1% TFA at a flow rate of 2 mL/min. The eluent was monitored for radioactivity and UV absorbance at 230 nm (t_{R}=7.5 min for [¹¹C]glyburide). The molar activity for [¹¹C]glyburide was determined by counting an aliquot of the product solution in a dose calibrator for radioactivity and integration of the UV peak associated with the radioactive peak for comparison with a pre-determined calibration curve of glyburide. Identity of the radioactive species was confirmed by co-injection of the radioactive product with a sample solution of glyburide and co-elution of the UV and radioactive peaks.

The average radiochemical yield of [¹¹C]glyburide was 5.7% based on trapped [¹¹C]methyl triflate activity, with radiochemical purity of >98% and average molar activity of 22.5 Ci/μmol at the end of synthesis (n=2).

PET Scan

Rats were sedated with isoflurane (3%) in a sedation chamber and kept anesthetized with isoflurane (1.5-2.5%). PET images were acquired using the Siemens FOCUS 220 PET scanner (Siemens Preclinical Solutions, Knoxville, Tenn.) with a reconstructed image resolution of ˜2 mm. Following a transmission scan, ¹¹C-glyburide was injected intravenously. List-mode data were acquired and dynamic scan data were reconstructed with a filtered back projection algorithm with corrections for attenuation, normalization, scatter and randoms. The left and right brain regions of interest (ROIs) were manually drawn based on the PET image. Regional time-activity curves (TACs) were generated for the left and right brain hemispheres.

### Results

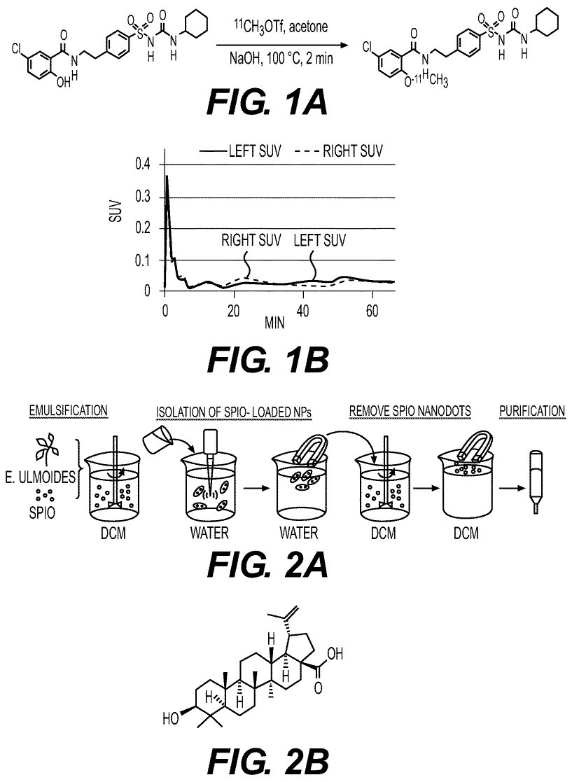

Glyburide has a limited ability to penetrate the ischemic brain. It was found that, despite the presence of stroke (confirmed by TTC staining), there was no significant difference in ¹¹C-glyburide uptake between the ischemic and the contralateral hemispheres (FIG. 1B), indicating that glyburide is unable to efficiently penetrate the ischemic brain.

### Example 2: Identification of BA as a Nanoparticle Forming Material

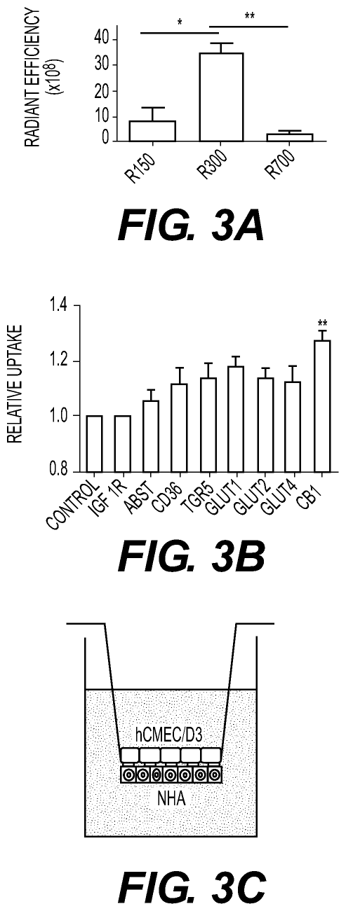

A chemical extraction approach was developed and used to test the hypothesis that certain medicinal herbs contain natural nanomaterials by analyzing E. ulmoides. In order to isolate nanomaterials that enable agent encapsulation, hydrophilic superparamagnetic iron oxide (SPIO) nanodots, (Strohbehn, et al. Journal of neuro-oncology 2015, 121 (3), 441-9) were used as the payload (FIG. 2A).

### Materials and Methods

As the first step, an extract of E. ulmoides was prepared by soaking it in dichloromethane (DCM), following by filtration. Next, the extract was emulsified with SPIO. SPIO-encapsulated NPs were then collected using a magnet. Successful encapsulation of SPIO was confirmed by transmission electron microscope (TEM).

After lyophilization, the SPIO-encapsulated NPs were dissolved in DCM. Free SPIO were removed by magnetization. The resulting extractant was separated using column chromatography. Different fractions were evaluated for NP formulation and characterized by thin layer chromatography (TLC). The TLC analysis was performed on the DCM extract (1), crude materials that enable SPIO encapsulation (2), and the selected material obtained after chromatography purification (3). TLC condition chloroform: methanol=95:5 (v/v); Chromogenic reagent: alcoholic solution of sulfuric acid (5%).

Identification of Betulinic Acid

E. ulmoides powder (50 g) was soaked in 400 mL of DCM for two days. After filtration, the DCM extract was obtained and emulsified with SPIO nanodots using the standard emulsion procedures as described by Han, et al. ACS nano 2016, 10 (4), 4209-18; Zhou, et al. Nat Mater 2012, 11 (1), 82-90. SPIO-encapsulated NPs were collected using a magnet. After lyophilization, SPIO-encapsulated NPs were re-dissolved in DCM. SPIO nanodots were removed using magnetic force. From these procedures, materials allowing for agent encapsulation were obtained. The resulting materials was separated using a silicon column (solvent: CHCl₃:MeOH, 97:3, v/v), different fractions were evaluated for NP formulation. One compound was obtained. ¹H-NMR, ¹³C-NMR, and mass spectrometry analyses identified it to be BA.

Transmission Electron Microscopy (TEM)

NPs resuspended in 10 μL water were applied to holey carbon-coated copper grids (SPI, West Chester, Pa., USA). A filter paper was used to absorb the NPs after 5 min. The grids were left at fume hood until completely dried and then visualized by using a JEOL 1230 transmission electron microscope (JEOL Ltd., Japan) at 100 kV.

### Synthesis of BA NPs

BA NPs were synthesized using the standard emulsion procedures (Han 2016; Zhou 2012). For typical synthesis of BA NPs encapsulated with hydrophobic cargos, including SPIO, IR780, and Glyburide, the selected cargo was dissolved together with 5 mg BA in mixed organic solution of DCM (0.95 ml) and methanol (0.05 ml), and added dropwise to a solution of 4 ml 2.5% PVA (aqueous phase). The resulting emulsion was sonicated on ice for 40 s (5 s on, 5 s off) and added to a stirring solution of 0.3% PVA in water (aqueous phase, 50 ml). After evaporation at 4° C. overnight, BA NPs were collected by centrifugation at 18,000 rpm for 30 min. Then, the pellets were suspended with 40 ml of water, and collected by centrifugation at 18,000 rpm for 30 min to obtain the NP pellets. Finally, the pellets were suspended with 5 ml of water, sonicated for 3 min, and then lyophilized for storage.

Scanning Electron Microscopy (SEM)

Samples were mounted on carbon tape and sputter-coated with gold, under vacuum, in an argon atmosphere, using a sputter current of 40 mA (Dynavac Mini Coater, Dynavac, USA). SEM imaging was carried out with a Philips XL30 SEM using a LaB electron gun with an accelerating voltage of 10 kV. The mean diameter and size distribution of the particles were determined by image analysis using image analysis software (ImageJ, National Institutes of Health). These micrographs were also used to assess particle morphology.

### Results

One compound was obtained, identified as BA (FIG. 2B) by ¹H-NMR, ¹³C-NMR, and mass spectrometry. Through the standard emulsion procedures, BA formed rod-shaped NPs in length of ˜315 nm and diameter of ˜60 nm, or 315(1)×60(d) nm, as determined by scanning electron microscope (SEM).

### Example 3: BA NPs for Delivery to the Ischemic Brain

### Materials and Methods

BA NPs were synthesized using DCM as the solvent, water as the aqueous phase, and 4° C. as the evaporation temperature, as described in Example 2. The shape and size of BA NPs were tunable by varying the organic phase, aqueous phase, and evaporation temperature. When a combination of ethyl acetate (EA) (solvent), water (aqueous phase), and 4° C. (evaporation temperature) was used, BA NPs were obtained with a size of 156(1)×45(d) nm as demonstrated by SEM imaging. When a combination of EA (solvent), NaOH solution (aqueous phase), and 25° C. (evaporation temperature) was used, BA NPs were obtained with a size of 730(1)×35(d). To simplify the nomenclature, BA NPs in the size of 156(1)×45(d) nm, 315(1)×60(d) nm, and 730(1)×35(d), were referred to as R150, R300, and R700, respectively.

R150, R300, and R700 were evaluated for delivery to the ischemic brain. NPs were synthesized with encapsulation of IR780, a near-infrared dye, and administered intravenously to MCAO mice. The amount of R150, R300, or R700 given to each mouse was normalized to ensure each received the same amount of fluorescence. After 24 hours, mice were euthanized. The brains were harvested and imaged.

Fluorescent Imaging

Mice with successful MCAO surgery were prepared. Immediately after surgery, IR780-loaded BA NPs were administered intravenously through the tail vein. Doses for each group were adjusted according to the fluorescence intensity to ensure that each mouse received the same amount of dye. Twenty-four hours later, mice were sacrificed to isolate the brain and other organs, and imaged by IVIS imaging system (Xenogen) with excitation wavelength of 745 nm and emission wavelength of 820 nm for free IR780 or IR780-loaded NPs. Fluorescence intensity in each brain was quantified using Living Image 3.0 (Xenogen).

### Results

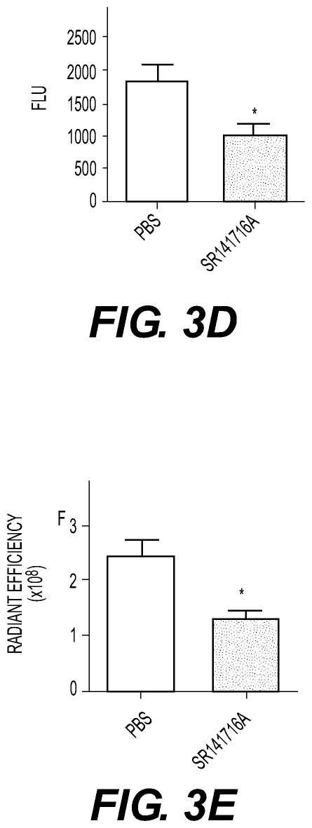

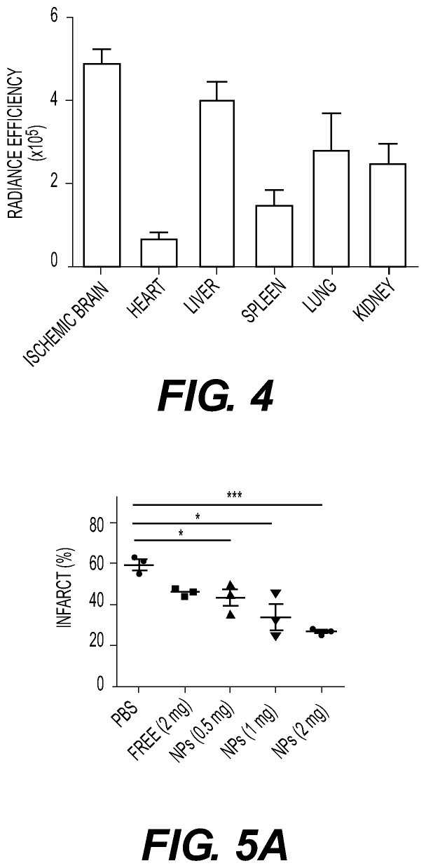

It was found that, among the three tested NPs, R300 demonstrated the greatest efficiency to accumulate in the ischemic region (as demonstrated by fluorescent imaging), which is four times and 10 times greater than R150 and R700, respectively (FIG. 3A). Biodistribution analysis showed that the accumulation of R300 in the brain was 1.2-fold greater than that in the liver (FIG. 4). In addition to the high efficiency, R300 also demonstrated a great specificity to the ischemic region: the location of ischemia identified by triphenyltetrazolium chloride (TTC) staining (white) well overlapped with the location of NPs detected based on fluorescence of cargo IR780 (red to yellow). Based on those result, R300 were selected for further investigation and referred as BA NPs.

### Example 4: Identification of Transporters/Receptors

### Materials and Methods

It was then examined if any transports or transports mediate the penetration of BA NPs into the brain. Analysis by MetaDrug (Thomson Reuters) predicted that BA may interact with several surface molecules, including insulin like growth factor 1 receptor (IGF-1R), apical sodium-bile acid transporter (ASBT), CD36, TGR5, glucose transporters (GLU1, 2, 4), and cannabinoid receptor 1 (CB1). To determine if any of them interact with BA NPs, candidate molecules in HEK293 cells, which were incubated with BA NPs encapsulated with coumarin 6 (C6), were overexpressed. Twenty four hours later, cells were collected. The uptake of BA NPs in cells was determined by flow cytometry.

To study the role of CB1 in NP transcytosis, a Transwell system was established as an in vitro model of the BBB by seeding astrocytes and endothelial cells on the basolateral and apical side, respectively. When transepithelial/transendothelial electrical resistance (TEER) values reached around 100Ω, SR141716A, a cannabinoid CB1 receptor blocker, was added to the upper chambers. One hour later, C6-loaded BA NPs were added. After 24 hours, the amount of NPs in the medium in the bottom chamber was determined.

Cell Culture

HEK293 cells were obtained from American Type Culture Collection (ATCC). Cells were maintained in DMEM supplemented with 10% v/v fetal bovine serum and PSG, all from Thermo Fisher, in a pre-humidified atmosphere at 37° C. containing 5% v/v CO₂.

In Vitro BBB Model and In Vitro Inhibition Study

After in vitro BBB model was successfully set up, upper chamber cells were pre-treated with CB1 inhibitor SR141716A (1 μM) or vehicle solution for 1 hour, then Coumarin 6 load BA NPs (100 μg/ml) were added into the upper chamber. 100 μl medium in lower chamber were taken out at 1 h, 2 h, 4 h, 8 h, and 24 h, the total amounts of dye were quantified based on fluorescence using a BioTek microplate reader.

In Vivo Blocking Study

Mice with successful MCAO surgeries were randomly divided into 2 groups (n=3 for each group), which received treatment of PBS and SR141716A, respectively. Thirty minutes later, IR780-loaded BA NPs were administered intravenously through the tail vein. Twenty-four hours later, mice were sacrificed to isolate the brain and imaged as above.

### Results

Results in FIG. 3B showed that among all cells, cells overexpressed with CB1 demonstrated the greatest efficiency. This result suggested that CB1 receptor, which is primarily expressed in the central nervous system, may mediate the transport of BA NPs into the brain.

FIGS. 3D and 3E show the role of CB1 mediating the transport of BA NPs into the brain. (D) In vitro analysis of the inhibitory effect of SR141716A on NP transcytosis. (E) Semi-quantification of IR780-loaded BA NPs in the brains isolated from MCAO mice with and without pre-treatment of SR141716A. Intensities of IR780 fluorescence were quantified using Living Image 3.0. Pre-treatment with SR141716A inhibited the transcytosis of BA NPs by 44%. To further confirm the finding, stroke mice were administered intravenously SR141716A. Thirty minutes later, mice were treated with IR780-loaded BA NPs. After an additional 24 hours, the accumulation of BA NPs in the brain was imaged and quantified based on the fluorescence of IR780. Consistent with the in vitro finding, blockade of CB reduced the uptake of BA NPs by 34% (FIG. 3E).

Taken together, these data indicate that BA NPs efficiently penetrate the ischemic brain, and the penetration efficiency is determined by their physical properties including size and shape, as well as the interaction with CB1.

### Example 5: Effect of Glyburide Loaded BA NPs on Stroke

### Materials and Methods

BA NPs were tested as a carrier for intravenous delivery of glyburide for stroke treatment. BA NPs were synthesized with encapsulation of glyburide. Glyburide is a potent agent for stroke treatment. On the other hand, glyburide, as a diabetes medication, may induce hypoglycemia at a high dose. Therefore, the loading of glyburide in BA NPs was limited to 0.005% by weight. The resulting NPs, termed as Gly-NPs, were characterized for physical properties and agent release.

MCAO mice were established and received intravenous administration of Gly-NPs at a dose equivalent to 5 μg/kg of glyburide per injection 0, 24, and 48 h after surgery.

In Vitro Agent Release

Gly-BA NPs (3 mg) were suspended in 1 mL buffer and incubated at 37° C. with gentle shaking. At each sampling time, NPs were centrifuged for 10 min at 12,000 rpm. The supernatant was collected and 1 mL buffer was added for continuously monitoring of the release. The amount of glyburide in supernatant was quantified by HPLC.

Determination of the Therapeutic Benefits

Mice with successful MCAO surgery were randomly divided into 4 groups (n=5 for each group), which received treatment of PBS, blank BA NPs, Gly-NPs at a dose equivalent to 5 μg/kg of glyburide, and the same amount of free glyburide, respectively. Mice were given treatment intravenously at 0, 24 and 48 h after surgery. Mice were monitored for survival for 10 days and were euthanized if one of the following criteria was met: (1) the mouse's body weight dropped below 15% of its initial weight, or (2) the mouse became lethargic or sick and unable to feed. For the study to determine the impact of treatments on infarct volume and neurological score, a cohort of mice were prepared (n=5) and received the same treatments as described above. Three days later, the score of each mouse was assessed by a standard behavioral test and were scored as follows: (1) normal motor function, (2) flexion of torso and contralateral forelimb when animal was lifted by the tail, (3) hemiparalysis resulting in circling to the contralateral side when held by tail on flat surface, but normal posture at rest, (4) leaning to the contralateral side at rest, and (5) no spontaneous motor activity. Therapeutic evaluations were carried out using an unbiased approach; the reviewer who scored mouse function was unaware of which treatment group each mouse belonged to. After the evaluations, the mice were sacrificed and the brains were excised, sectioned, and stained with TTC to determine the infract volume as described above.

Statistical Analysis

All data were collected in triplicate and reported as mean and standard deviation. Comparison between the groups were performed using a t-test. One-way ANOVA was used to analyze multiple comparisons by GraphPad Prism 7.0. *P<0.05, **P<0.01 and ***P<0.001 were considered significant.

### Results

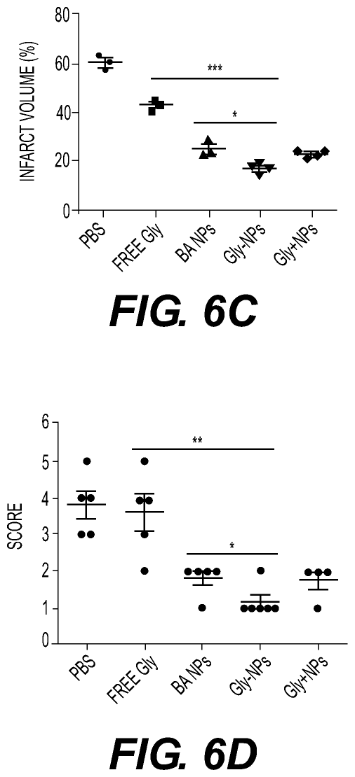

Analysis by SEM showed that encapsulation of glyburide did not alter the morphology of BA NPs. A controlled release study found that 91% of glyburide was released from Gly-NPs over three days (FIG. 6A).

Treatment with Gly-NPs significantly improved mouse survival (p<0.01, FIG. 6B), reduced infarct volumes by 36% (FIG. 6C) and improved neurological scores (FIG. 6D).

In contrast, treatments with the same amount of BA NPs or glyburide alone showed significantly less efficacy. The therapeutic benefits of Gly-NP treatment could be achieved simply through treatment with a mixture of the same amount of glyburide and BA NPs (Gly+NPs) (FIG. 6C, D), indicating that formulation in NPs is indispensable. Treatment with Gly-NPs significantly reduced brain infarct.

### Example 6: BA NPs Promote Stroke Recovery as an Antioxidant Agent

### Materials and Methods

BA NPs were evaluated for stroke treatment. Stroke mice were established and received an intravenous injection of BA NPs at 0.5, 1, or 2 mg at 0, 24, and 48 hours after surgery. At day 4, the mice were euthanized.

The brains were isolated and subjected to TTC staining.

Determination of the Therapeutic Benefits

For characterization of the treatment with BA NPs, mice with successful MCAO surgeries were randomly divided into 4 groups (n=3), which received treatment of PBS, Free BA, 0.5 mg BA NPs, 1.0 mg BA NPs and 2.0 mg BA NPs, respectively, at 0, 24 and 48 h after surgery. Three days later, the mice were sacrificed and the brains were excised, sectioned, and stained with TTC. The infarct area in each slice was quantified using ImageJ. The infarct volume was calculated by the formula described as: corrected infarct volume (%)=(contralateral hemisphere volume−non-infarcted ipsilateral hemisphere)/contralateral hemisphere volume×100.

Cignal™ Reporter Assay for Nrf2 Activity

Luciferase-based Nrf2 activity reporter and control constructs were obtained from Qiagen and co-transfected with Renilla luciferase-expressing construct pGL4.74 (Promega) to HEK293 cells using Fugene 6 transfection reagent (Promega). After treatment with BA NPs (100 μg/ml) for 48 hours, expression of firefly and Renilla luciferase were determined using a DUAL-LUCIFERASE® Reporter Assay System kit (Promega). The activity of Nrf2 signaling in cells, which was measured by the intensity of firefly luciferase, was normalized based on the intensity of Renilla luciferase.

Western Blot

To determine the anti-oxidant effect of BA NPs on cells, normal human astrocyte cells were randomly divided into 4 groups, which were treated with PBS, 2 μg/ml BA NPs, 10 μg/ml BA NPs and 30 μg/ml BA NPs. After 24 hours, cells were lysed in RIPA lysis buffer containing protease for 30 min on ice. The protein concentration of each cell lysate sample was determined using the BCA and adjusted to equivalent amounts. Western blot analysis was performed according to the standard procedures, using antibodies targeting Nrf2 (Novus Biologicals), HO-1 (Novus Biologicals), and beta-actin (#643802, BioLegend). To determine the anti-oxidant effect in vivo, mice with successful MCAO surgery were randomly divided into 2 groups (n=3 for each group), which received treatment of PBS or 2 mg BA NPs, respectively. After 24 hours, the brains were harvested, and the right hemispheres containing the ischemic area were excised. The brains from normal mice without surgery were used as controls. Western blot analysis was performed as described above.

### Results

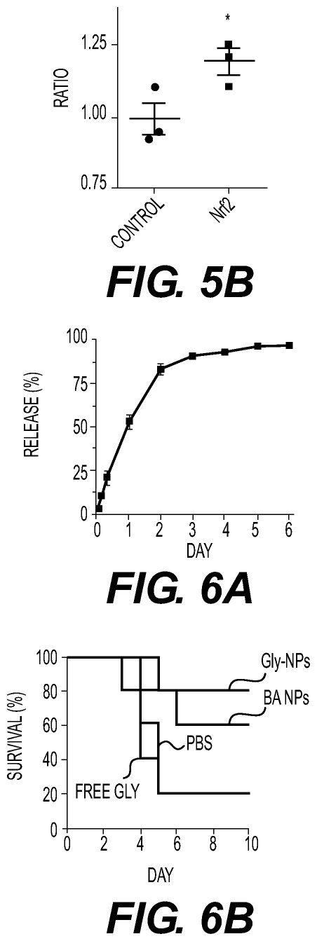

Results in FIG. 5B indicated that treatment with BA NPs significantly elevated the activity of Nrf2 signaling. Western blot analysis of BA NP-treated astrocytes and ischemic brain tissues showed treatment with BA NPs significantly up-regulated the expression of both Nrf2 and heme oxygenase-1 (HO-1), a Nrf2-regulated antioxidant enzyme.

Collectively, these results show that systemic treatment with BA NPs promoted stroke recovery through regulation of the antioxidant pathway.

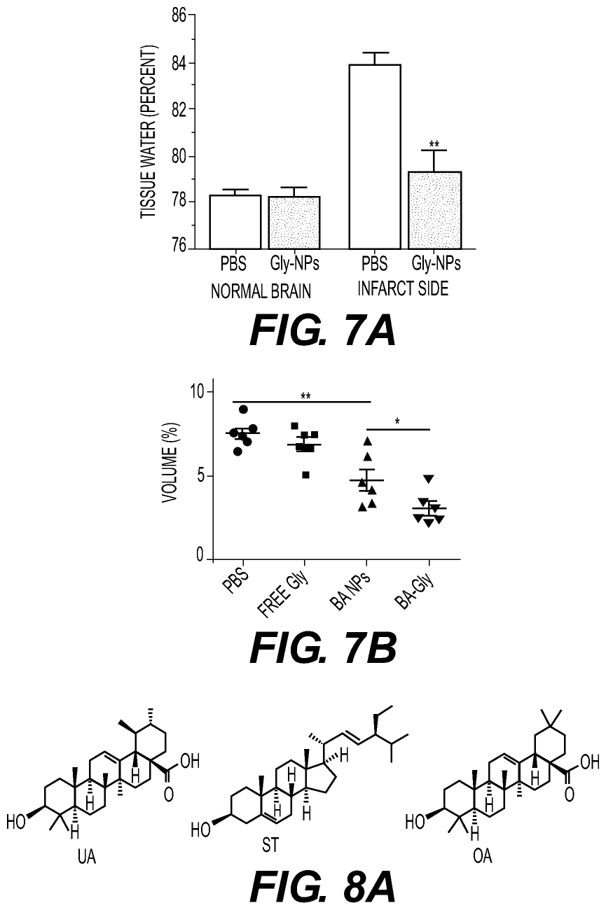

Glyburide-loaded BA NPs significantly reduced injured volumes in TBI mouse model, brain images and plot of brain volume (percent) for control PBS, free glyburide, BA NPs, and glyburide-loaded BA NPs (FIG. 7B).

Results showed that intravenous administration of BA NPs effectively reduced brain edema (FIG. 7A) and the infarct volume in a dose-dependent manner and reduced the infarct volume by 54% at the dose of 2 mg in the stroke mouse model, as shown in FIG. 5A.

### Example 7: Isolation of Antioxidant Nanomaterials from Other Herbs

### Materials and Methods

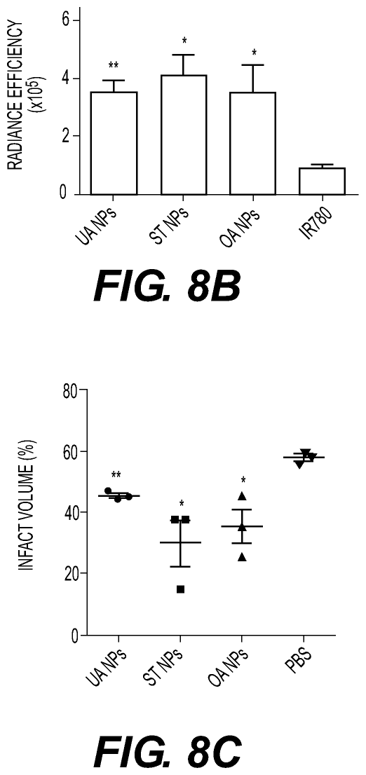

To exclude the possibility that the presence of antioxidant nanomaterials is unique to E. ulmoides, the chemical extract approach that was developed (FIG. 1A) was used to investigate a group of medicinal herbs, Eriobotrya japonica Thunb, Ophiopogon japonicas, and Olea europaea L, which were often used for management of antioxidant or anti-inflammation. Through this screen, UA, ST, and OA, which formed spherical or rod-shaped NPs (FIG. 8A), were identified. UA-, ST-, and OA-NPs were characterized in MCAO mice for brain penetration. UA-, ST-, and OA-NPs were synthesized with encapsulation of IR780, and intravenously administered into mice. After 24 hours, mice were euthanized. The brains were isolated and imaged.

### Results

Results in FIG. 8B show that, similar to BA NPs, all of them penetrated the ischemic brain in efficiency significantly greater than free dye. Similar to BA, UA, ST, and OA are known to have antioxidant activities, as reported by Nascimento, Molecules 2014, 19 (1), 1317-27; Yoshida, et al. J Nutr Sci Vitaminol (Tokyo) 2003, 49 (4), 277-80; Wang, et al. Chem Biol Interact 2010, 184 (3), 328-37. Next, UA-, ST-, and OA-NPs were accessed for promotion of stroke recovery using the same experiment procedures that were described above for evaluation of BA NPs. Results in FIG. 8C showed that, similar to BA NPs, all the tested NPs after intravenous administration significantly reduced brain infraction. These results suggest that antioxidant nanomaterials widely exist in medicinal herbs and could be identified through the approach established in this study.

### SUMMARY

Glyburide is known to have a limited ability to penetrate the BBB, as reported by Tournier, et al. Aaps J2013, 15 (4), 1082-90; Lahmann, et al. PloS one 2015, 10 (7), e0134476. In this study, through a PET imaging approach, it was found that glyburide is no more efficient in penetrating the brain on the ischemic side versus the ipsilateral side (FIG. 1). This finding may explain the observation in a recently completed GAMES-RP trial that intravenous administration of glyburide, although it enhanced patient survival, could not significantly improve clinical outcome, as reported by Sheth, et al. Lancet Neurol 2016, 15 (11), 1160-1169. To enhance the delivery of glyburide to the brain, a chemical extraction approach was developed to isolate BA, a natural nanoparticle forming material, from E. ulmoides. BA formed NPs, which were capable of penetrating the ischemic brain through interaction with CB1, improving functional recovery through antioxidant effects, and enhancing the delivery of glyburide to the brain for further improved efficacy. Other functional nanomaterials were isolated in medical herbs other than E. ulmoides.

This study is significant on two major fronts. First, the study demonstrates a method to discover functional nanomaterials from medicinal herbs for agent delivery. Different from most existing nanomaterials, such as polymers or lipids, which cannot penetrate the brain without further engineering and do not have biological activity without agent encapsulation, the nanomaterials isolated from medicinal herbs form NPs that may penetrate the brain and/or exhibit bioactivity. This finding may significantly impact drug delivery research through diversification of functional nanomaterials for drug delivery and disease treatment. The simplicity of these single-component NPs is beneficial for their clinical translation.

Second, this study establishes a new formulation of glyburide, Gly-NPs, which have several major advantages for stroke treatment. First, the dual acting NPs represent the current simplest solution to treat both cerebral edema and oxidation, two major complementary targets that are promising stroke treatment (Galgano, et al. Cell Transplant 2017, 26 (7), 1118-1130; Deb, et al. Pathophysiology 2010, 17 (3), 197-218). Second, the employment of BA NPs as the delivery vehicle not only enhances the delivery of glyburide to the brain, allowing full capitalization of glyburide as an anti-edema agent, but also reduces the side effect of glyburide. In a current clinic, the efficacy of glyburide has been limited by a low dose (3 mg/d), as glyburide given at higher doses may induce hypoglycemia. The use of BA NPs reduces the exposure of glyburide to the circulatory system and thus limits the risk of hypoglycemia. Third, the employment of BA NPs makes it convenient to deliver glyburide to patients. Due to its limited brain retention and short plasma half-life, current use of glyburide requires continuous infusion for 72 hours. As reflected in preclinical animal studies, glyburide required continuous administration using osmatic pumps (Simard, et al. Nature medicine 2006, 12 (4), 433-40). Different from free agents, NPs have the sizes optimal for longer retention in brain tissue and can provide controlled release of cargo agents over time. It was found that daily injection of Gly-NPs is sufficient to generate adequate therapeutic benefit.

In summary, the problem of glyburide having a limited ability to penetrate the ischemic brain has been overcome using a new formulation of glyburide through encapsulation into BA NPs, which provides anti-edema and antioxidant combination therapy via the simplest formulations. Due to its simplicity, multifunctionality, and significant efficacy, the resulting formulation may be promptly translated into clinical applications to improve clinical management of stroke.

### Example 8: Preparation of Chemically Modified BA NPs for Acidity-Triggered Agent Release

### Materials and Methods

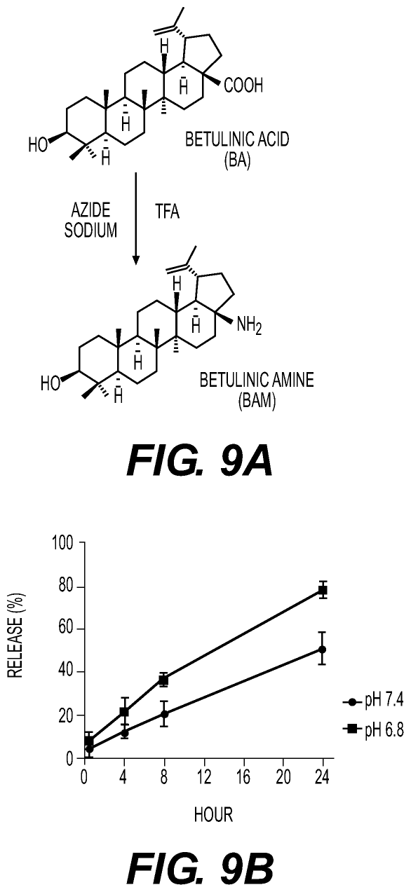

To promote agent release from BA-NPs, amine derivatives of BA were synthesized and characterized as shown in FIG. 9A.

### Results

BA-NPs are sensitive to alkaline pHs and mostly degraded in PBS buffer with pH 8.0 after overnight incubation. By contrast, NPs synthesized using betulinic amine (BAM), which are of similar morphology as BA-NPs, were stable in alkaline pHs but sensitive to acidic pHs (FIG. 9A). Consistently, BAM-NPs release cargo glyburide in a rate significantly greater than BA-NPs (FIG. 9B). Consistently, after overnight incubation in pH 6.8, most BAM-NPs lost their structure as demonstrated by SEM imaging.

### Example 9: AMD3100-Conjugated BAM-NPs for Improved Delivery to the Ischemic Brain

### Materials and Methods

The same methods and materials were used as described above to assess improved delivery to the ischemic brain.

Ma1-PEG2000-NHS was conjugated to BAM NPs through NHS-amine reaction to AMD3100 was activated with N,N′-cystaminebisacrylamide and conjugated to NPs as reported by Guo X. et al. ACS Nano, 2018, 12, 8723-8732. AMD3100 is a small molecule that binds CXCR4. It was used as a ligand for targeted delivery to a tissue following a stroke (Guo X. et al. ACS Nano, 2018, 12, 8723-8732).

For non-invasive imaging, nanoparticles were synthesized with encapsulation of IR780, an infra-red florescence dye. The radiance efficiency was measured to assess the delivery of NPs to the brain. Radiance is the florescence unique when images were acquired and quantified by the IVIS Spectrum In Vivo Imaging System.

The infarct volume (percent) was measured to assess the efficacy of peptide therapeutic Tat-NR2B9c for stroke treatment, comparing control PBS, Tat-NR289c (3 nM/g), NPs, Tat-NR289c-NPs (3 nM/g), Tat-NR289c-NPs (1 nM/g) and Tat-NR289c-NPs (0.5 nM/g).

### Results

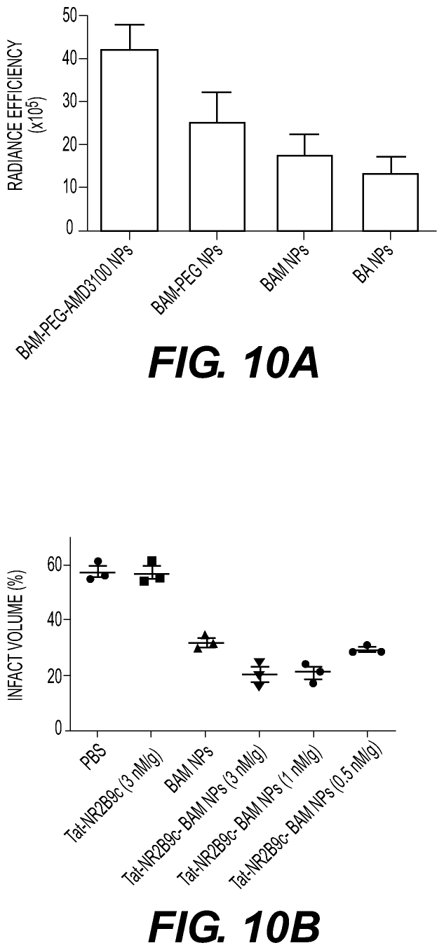

As shown in FIG. 10A, AMD3100-conjugated BAM-NPs improved the delivery of peptide therapeutic Tat-NR2B9c for stroke treatment relative to controls.

Quantification of infarct volumes in the brains isolated from MCAO mice received treatment of the indicated treatments demonstrated the therapeutic effect of Tat-NR2B9c-NPs in a dose-dependent manner (FIG. 10B).

### Example 10: Additional Nanomaterials

### Materials and Methods

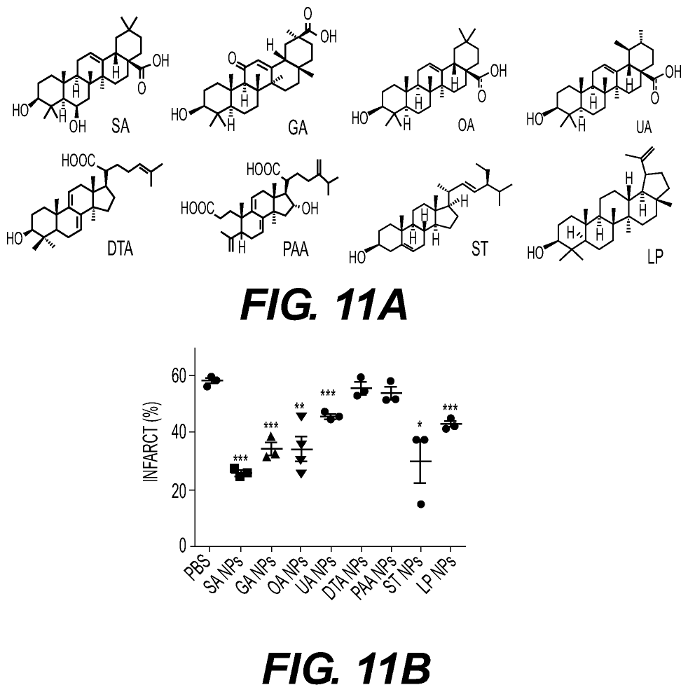

46 MNPs were screened, most of which are often used for the treatment of brain injuries in traditional medicine. Eight nanomaterials, including sumaresinolic acid (SA), glycyrrhetic acid (GA), oleanolic acid (OA), ursolic acid (UA), dehydrotrametenolic acid (DTA), poricoic acid A (PAA), lupeol (LP), and β-sitosterol (BT), were identified (FIG. 11A). Their activities in reducing stroke damage were also studies in MCAO mice by intravenously administering 2 mg of each type of NPs.

### Results

Among the eight nanomaterials, SA, GA, OA, US, and LP form spherical NPs and the rest form rod-shaped NPs. Table 1 summarizes the physiochemical properties and loading efficiency of the NPs.

NPs consisting of SA, GA, or OA can efficiently encapsulate glyburide at 58-65% loading efficiency (Table 1). NPs consisting of SA, GA, OA, and ST significantly reduced infraction in a degree that is comparable to BA NPs (FIG. 11B).

TABLE 1
physiochemical properties and loading efficiency of the NPs

| | | Average | Zeta- | Loading |
|---|---|---|---|---|
| | | size | potential | Efficiency |
| | Morphology | (nm) | (mV) | (%) |
| SA NPs | Sphere | 126.3 | −19.2 | 58.6 |
| GA NPs | Sphere | 218.2 | −25.2 | 65.3 |
| OA NPs | Sphere | 143.2 | −19.3 | 47.8 |
| UA NPs | Sphere | 138.6 | −18.2 | — |
| DTA | Rod | 101.3 × 423.5 | −20.1 | — |
| NPs | | | | |
| PAA | Rod | 81.7 × 420.3 | −20.8 | — |
| NPs | | | | |
| LP NPs | Sphere | 149.6 | −23.6 | — |
| ST NPs | Rod | 98.6 × 496.7 | −24.9 | — |

1. An injectable or topical therapeutic, prophylactic or diagnostic nanoparticulate formulation comprising a therapeutically, prophylactically or diagnostically effective amount of supramolecular particles, optionally comprising a therapeutic, prophylactic, nutraceutical or diagnostic agent, comprising a material selected from the group consisting of diterpene resin acids, phytosterols, lupane-type pentacyclic triterpenes, oleanane-type pentacyclic tritepenes, lanostane-type triterpenes and combinations thereof.

2. The formulation of claim 1 comprising
a plurality of one or more compounds defined by formula 1, 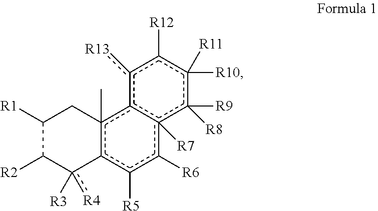
and optionally a therapeutic, prophylactic, or diagnostic agent,
wherein the compounds are associated with one another via non-covalent interaction comprising hydrogen-bonding interaction, π-π interaction, or solvophobic-solvophobic interaction;
wherein R1 is H, OH, or C(═O)R16; R2 is H or R17; R3 is H, CH₃, or R18; R4, if single bonded, is H, CH₃ or R19, or R4, if double bonded, is CH₂; R5 is H or OH; R6 is H or OH; R7 is H or CH₃; R8 is H or CH₃; R9 is H or R14; R10 is R15 when R9 is R14, or R10 is R20 when R9 is H; R11 is H, CH₃, or R21; R12 is H or OH; R13, if single bonded, is H, or R13, if double bonded, is O or S; R14 and R15 combine to form a five-membered ring, a six-membered ring, or a six-membered ring fused with another five-membered or six-membered ring;
R16, R17, R18, R19, R20, or R21 are individually a derivatizing group comprising an amine, a polyethylene glycol, OH, a carboxyl, an alkyl, an alkene, an amide, a sulphonyl, an aryl, a carbohydrate, or a combination thereof;
wherein each dashed line between two atoms otherwise connected by a solid line indicates, individually, the two atoms are monovalently connected or divalently connected, the number of divalently connection not exceeding allowed valency in fused cyclic rings; and wherein the dash line between two atoms not otherwise connected by a solid line indicates a monovalent bond or no covalent bond.

3. The formulation of claim 2, wherein R1 is C(═O)R16; R2═R3═R5═R6═R7═R12═H; R13 is single bonded and is H; R4 is double bonded and is CH₂; R8═R11═CH₃; R9 is R14; R10 is R15; R14 and R15 combine to form a five-membered ring; and the compounds are defined by Formula 2: 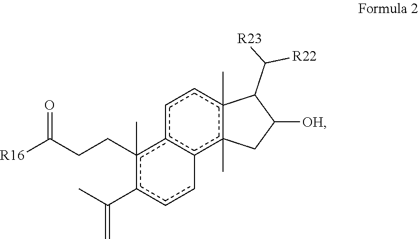
wherein R22 and R23 are individually a derivatizing group comprising a carboxyl, an alkyl, an alkene, a poly(ethylene glycol), an amine, OH, or a combination thereof.

4. The formulation of claim 3, wherein the compounds are poricoic acid A, poricoic acid AE, derivatives thereof, or a combination thereof.

5. The formulation of claim 2, wherein R1═R5═R6═R7═R12═H; R2═OH or R17; R3 is H or CH₃; R4 is H or CH₃; R9 is R14; R10 is R15; R14 and R15 combine to form a five-membered ring; R11 is CH₃; R13 is single bonded and is H; and the compounds are defined by Formula 3: 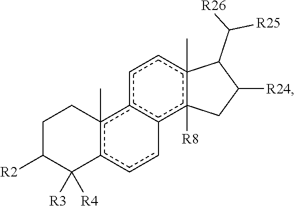
wherein R24 is H or OH; R25 and R26 are individually a derivatizing group comprising a carboxyl, an alkyl, an alkene, a poly(ethylene glycol), an amine, OH, or a carboxyl with the hydrogen replaced by

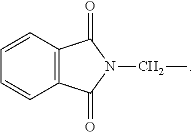

6. The formulation of claim 5, wherein the compounds are dehydrotrametenolic acid, pachymic acid, beta sitosterol, cholesterol, ergosterol, campesterol, stigmasterol, derivatives thereof, or a combination thereof.

7. The formulation of claim 2, wherein R1═R3═R4═R5═R7═R8═R13═H; R11 is CH₃; and the compounds are defined by formula 4: 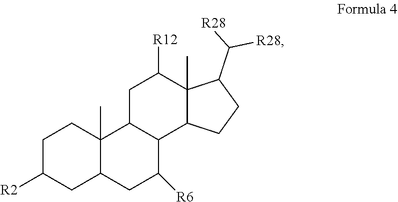
wherein R27 and R28 are individually a derivatizing group comprising a carboxyl, an alkyl, an alkene, a poly(ethylene glycol), an amine, an amide, OH, a sulphonyl.

8. The formulation of claim 7, wherein the compounds are cholic acid, glycocholic acid, taurocholic acid, deoxycholic acid, lithocholic, glycochenodeoxycholic acid, taurochenodeoxycholic acid, ursodeoxycholic acid, chenodeoxycholic acid, derivatives thereof, or a combination thereof.

9. The formulation of claim 2, wherein R1═R2═R5═R6═R7═R8═R9═R12═R13═H; and the compounds are defined by formula 5: 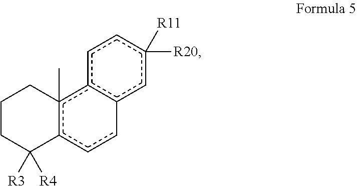
wherein R3, R4, R20 and R11 are individually a derivatizing group comprising a carboxyl, an alkyl, an alkene, a poly(ethylene glycol), an amine, an amide, a sulphonyl, OH, or a combination thereof.

10. The formulation of claim 9, wherein the compounds are isopimaric acid, abietic acid, dehydroabietic acid, isodextropimaric acid, derivatives thereof, or a combination thereof.

11. The formulation of claim 2, wherein R1 is H or OH; R4═R7═R8═CH₃; R6 ═R11═R12═H; R9 is R14; R10 is R15; R14 and R15 combine to form a six-membered ring fused with another five-membered ring; the compounds are defined by Formula 6: 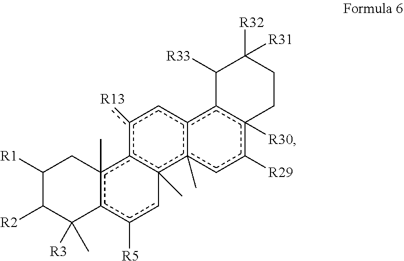
wherein R29 is H or OH; R30, R31, R32, and R33 are individually a derivatizing group comprising a carboxyl, an alkyl, an alkene, a poly(ethylene glycol), an amine, an amide, OH, a sulphonyl, or a combination thereof.

12. The formulation of claim 11, wherein the compounds are oleanolic acid, ursolic acid, sumaresinolic acid, echinocystic acid, maslinic acid, beta-boswellic acid, glycyrrhetic acid, glycyrrhizic acid, derivatives thereof, or a combination thereof.

13. The formulation of claim 2, wherein R1═R5═R6═R11═R12═R13═H;
R7═R8═CH₃; R9 is R14; R10 is R15; R14 and R15 combine to form a six-membered ring fused with another five-membered ring; the compounds defined by formula 7: 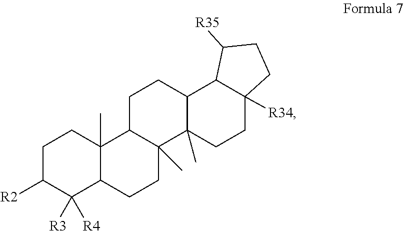
wherein R34 and R35 are individually a derivatizing group comprising a carboxyl, an alkyl, an alkene, a poly(ethylene glycol), an amine, an amide, OH, a sulphonyl, or a combination thereof.

14. The formulation of claim 13, wherein the compounds are lupeol, betulinic acid, betulin, derivatives thereof, or a combination thereof.

15. The formulation of claim 1, wherein the particles are in a topical excipient selected from the group consisting of lotions, gels, powders, creams, aerosols, and sprays.

16. The formulation of claim 1, wherein the particles are formulated in a sterile aqueous excipient for injection.

17. The formulation of claim 1, comprising therapeutic, prophylactic, or diagnostic agent encapsulated or incorporated into the particles at between about 0.5% and about 50%, preferably between about 5% and 30%, by weight.

18. The formulation of claim 1, wherein the particles have an average diameter between about 10 nm and 300 nm.

19. The formulation of claim 1 comprising a compound selected from the group consisting of betulinic acid, ursolic acid, stigmasterol, and oleanolic acid.

20. The formulation of claim 1 effective for the treatment or prevention of stroke, ischemic damage, oxidative stress, excitotoxicity, inflammation, platelet aggregation, edema, or imaging tissue associated therewith.

21. The formulation of claim 20 comprising an agent selected from the group consisting of glyburide, butylphthalide, NA1, fingolimod, and ticagrelor.

22. The formulation of claim 20 comprising betulinic acid and glyburide.

23. A method of treatment, prevention or diagnosis for stroke, ischemic damage, traumatic brain injury, oxidative stress, excitotoxicity, inflammation, platelet aggregation, edema, or imaging tissue associated therewith comprising administering the formulation of claim 20.

24. Use of the formulation of claim 20 for the treatment, prevention or diagnosis for stroke, ischemic damage, traumatic brain injury, oxidative stress, excitotoxicity, inflammation, platelet aggregation, edema, or imaging tissue associated therewith.
